(19) United States
(12) Patent Application Publication
(10) Pub. No.: US 2019/0309026 A1
(43) Pub. Date: Oct. 10, 2019

(54) Compositions and Methods for Altering Amyloid Precursor Protein (APP) Processing

(71) Applicant: DREXEL UNIVERSITY, Philadelphia, PA (US)

(72) Inventors: Olimpia MEUCCI, Penn Valley, PA (US); Renato BRANDIMARTI, Bologna (IT)

(21) Appl. No.: 16/380,719

(22) Filed: Apr. 10, 2019

Related U.S. Application Data
(60) Provisional application No. 62/655,537, filed on Apr. 10, 2018.

(51) Int. Cl. C07K 14/005; C12N 9/64; A61P 25/28

(52) U.S. Cl. CPC C07K 14/005; C12Y 304/24081; C12N 9/6489; A61K 38/00; C07K 2319/03; C12N 2710/16033; A61P 25/28

(57) ABSTRACT

The present invention relates to the discovery of compositions and methods for altering Amyloid Precursor Protein (APP) processing. Alerting APP processing is aids the treatment of neuropathological disorders such as those associated with HIV infection and Alzheimer's disease (AD). The invention includes fusion protein constructs that include an effector protein and an HSV US9 protein or functionally active fragment thereof that reduce the amount of amyloid β-protein produced in a cell.

### CROSS-REFERENCE TO RELATED APPLICATIONS

The present application claims priority under 35 U.S.C. § 119(e) to U.S. Provisional Application No. 62/655,537, filed Apr. 10, 2018, which is hereby incorporated by reference herein in its entirety.

### STATEMENT REGARDING FEDERALLY SPONSORED RESEARCH OR DEVELOPMENT

This invention was made with government support under grant numbers DA040519 and DA015014 awarded by the National Institutes of Health (NIH). The government has certain rights in the invention.

### BACKGROUND OF THE INVENTION

Despite effective antiretroviral treatment, cognitive dysfunction is present in up to 50% of individuals infected with HIV. This dysfunction—collectively known as HIV associated neurocognitive disorder (HAND)—can be enhanced in the aged population, which represents a sizeable and growing proportion of the HIV+ community. Converging clinical and preclinical evidence suggest that specific biochemical and structural changes in neurons may similarly underlie cognitive deficits in both HAND and aging (normal and pathological such as in Alzheimer's disease (AD)). For example, both HIV proteins and aging increase plasma membrane ceramide content, which can profoundly alter membrane signaling events by differentially segregating membrane proteins and driving their internalization. As a result, membrane ceramide can amplify inflammatory signaling (IL1β, TNFα) and processing events (Amyloid Precursor Protein, APP, to Amyloid-β) that are frequently triggered by infection and CNS injury, and are strongly implicated in both HIV and age-related cognitive dysfunction. Although many additional cellular and metabolic events have been implicated in the neuropathogenesis of Alzheimer's disease (AD), the misprocessing of APP and its dependence on lipid-rafts have been consistently reported as signature findings in AD.

Much research has been dedicated to the identification of novel therapeutic agents that can be used to effectively treat and/or prevent neurocognitive disorders. But there still exists a need to develop effective therapies for treating neurocognitive disorders, where APP misprocessing to Amyloid-β formation has been observed. The present invention meets this need.

### BRIEF SUMMARY OF INVENTION

The invention provides a construct comprising at least one effector protein or a functionally active fragment thereof and a Herpes Simplex Virus (HSV) US9 protein or a functionally active fragment thereof. The invention further provides an expression vector encoding the construct of the invention. The invention further provides a method of reducing levels of amyloid β-peptide in a subject. The invention also provides, a method of treating a neuropathological condition in a subject. Additionally, the invention provides a kit that includes a pharmaceutical composition containing the construct of the invention as well as instructional material for use thereof.

In certain embodiment, the effector protein or the functionally active fragment thereof is covalently coupled to the HSV US9 protein or a functionally active fragment thereof. In certain embodiments, the effector protein is a non-amyloidogenic Amyloid Precursor Protein (APP)-cleaving protease. In certain embodiments, the effector protein is a peptidase domain of A disintegrin and metalloproteinase (ADAM10). In certain other embodiments, the effector protein is a phosphotyrosine binding domain (PTB) of X11.

In certain embodiments, the construct/pharmaceutical composition of the invention advantageously reduce amyloid precursor protein's (APP's) β-amyloidogenic processing by promoting ADP's α-cleavage without adversely affecting any other cellular functions.

### BRIEF DESCRIPTION OF THE DRAWINGS

The following detailed description of specific embodiments of the invention will be better understood when read in conjunction with the appended drawings. For the purpose of illustrating the invention, the drawings illustrate specific embodiments. It should be understood, however, that the invention is not limited to the precise arrangements and instrumentalities of the embodiments shown in the drawings.

FIGS. 1A-1D illustrate topological organization and intracellular distribution of US9-driven fluorescent cargos. FIG. 1A shows electrophoretic analysis of proteins from 293T cells expressing gfp-US9 (g9). Cells were solubilized in the presence of cold Triton X100, and separated on Optiprep density gradient. The presence of g9 was detected on each fraction with an antibody against gfp (top gel) and compared to the distribution of the endogenous. Transferrin Receptor (TfR) in the bottom gel. Total cell lysates (L) patterns are shown on the first lane, and sequential fractions collected from top are loaded in lanes 1-12. gfp-US9 accumulation in fractions 5-6 indicates its association with lipid rafts. Fractions were loaded on two gels, as indicated by the vertical line between lanes 8 and 9 of each blot. The full-length blots are presented in FIG. 12. FIG. 1B shows schematic description of the US9 chimeric constructs generated through modifications of the N- and C-terminus. The US9 membrane orientation determines the exposure of gfp and mCherry on the cytosolic and lumenal/extracellular side of the membrane, respectively. FIGS. 1C and 1D are confocal images of rat neurons showing the intracellular localization of the US9-based fluorescent probes described in FIG. 1B. g9 and 9mC fluorescent patterns were analyzed in FIG. 1C, and their distributions merged in g9-9mC reveal a similar behavior of the two differently oriented constructs. A punctate staining is also obtained in cells expressing the double reporter g9mC (FIG. 1D), with a distribution indistinguishable from that of gfp-US9. Scale bar is 10 μm.

FIG. 2 shows membrane orientation of US9 C-terminal cargos. Rat neurons expressing 9mC were incubated with the anti-mCherry antibody, in the absence and in the presence of permeabilizing agents. The presence of mCherry on the non-cytosolic side of the membrane was revealed using a fluorescent secondary Ab, as represented in the schematic cartoon. The plasma membrane signal shown in the central panels indicates the correct orientation of the fluorescent molecule attached to the C-terminus of US9. The mCherry portion of 9mC (left panels) is not accessible to the antibody in non permeabilized cells, and no overlap between mCherry and anti-mCherry from 9mC in intracellular vesicles can be seen in the merged images on right panels. Insets show higher magnifications of the boxed region of the same cell. In permeabilized cells in bottom images, intracellular distribution of 9mC (revealed by the fluorescence on the left micrograph) overlaps the immunodetection obtained with the antibody against mCherry (central image; merged image on the right), at both the plasma membrane and intracellular vesicles. Scale bar is 10 μm (2 μm in insets).

FIGS. 3A-3B show US9-driven functional assay. (FIG. 3A) Schematic representation of elements used to generate the US9 functional assay. The elements were assembled as depicted in the bottom part of the cartoon on the left, with the cleaved product on the right. (FIG. 3B) Electrophoretic analysis of proteins extracted from 293T cells co-transfected with a constant amount of the substrate g9tcsmC and increasing amounts of the protease 9t, ranging from 0 to 2 μg. The same membrane was incubated with antibodies against HA, gfp, and mCherry to reveal the presence of the effector protein (9t), N-terminal cleaved product (gfp), and C-terminal cleaved fragment (mCherry), as lots are presented in FIG. 13.

FIGS. 4A-4B show US9-driven TEV protease expression correlates with substrate processing. (FIG. 4A) Electrophoretic analysis of proteins extracted from 293T cells co-transfected with a constant amount of the substrate g9tcsmC and increasing amounts (ranging from 0 to 150 ng) of the protease 9t. The presence of uncleaved and cleaved substrate was revealed with the anti-mCherry antibody. In this range of expression, the increasing presence of the US9-driven protease correlates with a constant decrease of the full length substrate and the corresponding increase of the processed fragment. The densitometric analysis of the same blot was used to generate the histogram shown in FIG. 4B. The height of the columns corresponds to the % of cleavage in each individual sample, calculated as described in Result from full-length blot is presented in the FIG. 14.

FIGS. 5A-5C depict membrane orientation dependence of the US9-driven functional assay. Novel US9-driven protease (t9mC) and substrate (gtcs9mC) were generated as schematically represented in (FIG. 5A) bottom part. In the new constructs, the cargos are attached to the N-terminus of US9 and are exposed on the cytosolic leaflet of membranes, resulting in an inverted orientation with respect to 9t and g9tcsmC. (FIG. 5B) Electrophoretic analysis of proteins extracted from 293T cells transfected as indicated. The substrate with TEV protease cleavage site confined in the vesicle lumen is accessible to 9t but not to t9mC, as revealed by the absence of the specific band reacting with the anti-mCherry antibody in the lane corresponding to cells co-transfected with g9tcsmC and t9mC. (FIG. 5C) The same cytosol-oriented protease is effective against the substrate with the cleavage site on the cytosolic leaflet of vesicle membranes, and the corresponding gfp product can be seen in cells coated with t9mC and gtcs9mC. The full-length blots are presented in the FIG. 15

FIGS. 6A-6B depict BACE1-based substrate for US9-driven functional assay. The sequence encoding amino acids 454-481 of BACE1, comprising the trans-membrane domain, was used to generate the US9 functional assay substrate gtcsBmC. In this construct, the TEV cleavage site is inserted between gfp and BACE1-TM, as represented in the cartoon in FIG. 6A. BACE1 is type 1 membrane protein, with the C-terminus exposed on the cytosolic side of membranes. The membrane orientation of the BACE1-based substrate, with respect to the other elements of the US9 functional assay, is schematically visualized in the boxed cartoon. In 293T cells expressing constant amounts of gtcsBmC, increasing presence of 9t results in the appearance of a cleaved product, revealed by the band corresponding to gfp in the electrophoretic analysis in FIG. 6B. Cleavage efficiency determined as already described correlates with the amount of 9t DNA transfected in each individual sample and is graphically rendered in the chart. The full-length blot is presented in FIG. 16.

FIGS. 7A-7D show US9-driven TEV protease activity on lipid raft and non lipid raft substrates. (FIG. 7A) Two APP-based substrates were generated as depicted, by inserting the APP TM domain (aa 700-723) into the substrate backbone. In gtcsAmC, the TEV protease cleavage sequence (tcs) is upstream of aa 694-729 of APP. In gtcsA695TMmC the APP sequence starts from amino acid 694 and goes to the C-terminus of APP, and comprises trans-membrane and cytosolic domains. The APP α-cleavage site (aa 688) is not present in the constructs. Canonical APP770 aa numbering is used here. (FIG. 7B) Non-lipid raft substrate and protease were based on the HSV glycoprotein C TM domain (aa 481-497), as schematically represented. (FIG. 7C) The orientation and lateral membrane organization of generated substrates and proteases is represented in the cartoon. US9 is a type 2, while BACE1, APP, and gC are type 1 membrane proteins. All constructs are designed and assembled in order to expose cleavage site and protease in the lumen of transport vesicles. Moreover, lateral proximity dependent on accumulation in lipid rafts is visually represented in the cartoon. (FIG. 7D) The ability of 9t to cleave substrates driven by the different TM domains indicated in (FIG. 7C) was determined in 293T cells. Targeting domains derived from BACE1 (lanes 3-4) and APP (lanes 5-6 for TM and 7-8 for TM+cytosolic domain) were all able to drive the TEV protease cleavage site in close proximity of 9t, resulting in effective processing as demonstrated by the appearance of the band corresponding to gfp (boxed region of the western blot). The substrate driven by gC TM domain was unaffected by the co-expression of 9t, as no difference is detectable between lanes 1 (gtcsCTMmC) and 2 (gtcsCTMmC+9t). The same substrate was readily cleaved in cells co-transfected with the gC TM targeted protease (lanes 9-10). The full-length blots are presented in the FIG. 17.

FIG. 8 shows membrane orientation of US9 N-terminal cargos. Confocal microscopy was used to image rat cortical neurons expressing g9 (: gfp: anti-HA; top row: non-permeabilized neurons; bottom row permeabilized neurons). As shown in the central micrographs, immunodetection of the HA epitope was precluded in non-permeabilized neurons, while the same epitope was readily available in permeabilized cells. Scale bar is 10 μm.

FIGS. 9A-9B show the US9-driven functional assay. FIG. 9A shows representative images of 293T cells co-expressing US9 with C-terminal cargos mCherry (9mC) and TEV protease (9t) are shown in the top panels. The distribution of 9mC is revealed by the mCherry signal in the left panel, while 9t localization (in the center) is detected with an antibody against the HA epitope present in 9t. Merged signal in the right image confirms the co-localization of the two molecules. The distribution of the substrate g9tcsmC is shown in the lower panels (mCherry; gfp; merged image). Scale bar is 10 μm. FIG. 9B shows quantitative analysis of cleavage efficiency. Densitometric quantification of the abundance of bands corresponding to uncleaved (black box) and cleaved (greybox) substrate in 293T cells, cotransfected as visually indicated on the top of the western blot, was used to assess the cleavage efficiency of the US9-driven TEV protease. Columns on the bottom chart represent the percentage of processing in each individual sample. The gfp antibody was used for detection.

FIG. 10 shows electrophoretic analysis of proteins from 293T cells expressing gtcsCTMmC, fractionated on a Optiprep gradient. The presence of gtcsCTMmC was detected on each fraction with an antibody against gfp. Sequential fractions collected from top are loaded in lanes 1-12. Samples were run on two gels, as indicated by the line between lanes 9 and 10 in the first blot (top). The full-length blots are also presented separately below the cropped blot (boxes mark lanes corresponding to the top blot).

FIG. 11 is a schematic showing recombinant proteins. With the exception of g9, all other constructs were generated in this study. On the left, the membrane orientation and composition of each recombinant protein is graphically rendered. On the right, the regions joining the different elements in the chimeras (gfp, mCherry, TEV protease, targeting domain) are expanded to show amino acid composition. The TEV protease cleavage site (tcs) is in bold, underlined. Cleavage occurs between Q and G. The HA epitope is in bold. Sequences for regions joining different elements (gfp, mCherry, TEV protease, targeting domain) of the construct correspond to SEQ ID NOs: 8-22 as provided elsewhere herein.

FIG. 12 shows original blots used to generate FIG. 1A.

FIG. 13 shows original blots used to generate FIG. 3B.

FIG. 14 shows original blots used to generate FIG. 4A.

FIG. 15 shows original blots used to generate FIGS. 5B and 5C; boxes mark the lanes reported in FIG. 5.

FIG. 16 shows original blots used to generate FIG. 6B.

FIG. 17 shows original blots used to generate FIG. 7D.

FIGS. 18A-18B show effect of g9A10pep on β-processing of APP. (FIG. 18A) The APP C-terminal fragment generated in APP-expressing HEK cells was detected using a amyloid-β-specific antibody that recognizes the ˜10 KDa cleaved product of APP after BACE 1 cleavage from cellular lysates. Overexpression of APP can induce the production of this β-cleaved APP fragment (lane 2), which is heavily enhanced by overexpression of BACE1 (lane 4). The presence of g9A10pep dramatically reduced the accumulation of the β-cleaved APP product (lanes 3 and 5). (FIG. 18B) Amyloid-3 peptides released from HEK cells were immunoprecipitated and detected using the same amyloid-β-specific antibody. The Amyloid-β peptides were present in the medium of cells in which the amyloidogenic pathway was induced by the presence of BACE1 (lane 4). The release of the cleaved product was almost completely abolished by the activity of g9A10pep (lane 5). This fragment is also absent in ADAM10 overexpressing cells (lane 6).

FIG. 19 is a schematic showing plaque formation. (Zhang, C., 2012. Discovery medicine, 14(76), 189-197).

FIGS. 20A-20D illustrate that US9-based molecular tools can reduce APP β-amyloidogenic processing by exploiting multiple molecular mechanisms. (FIG. 20A) Aβ seeding to toxic oligomers and plaques requires the APP combined cleavage by β- and γ-secretases. The alternative α-secretase cleavage results in non-toxic and likely neurotrophic and neuroprotective products. Also, interaction of APP with X11 proteins (through APP-Cterminal and X11 PTB domains) reduces β-secretase activity by dislocation of the protein from BACE1-enriched cellular districts. (FIG. 20B) ADAM10 is the major α-secretase involved in non-amyloidogenic processing of APP. The ADAM10 peptidase domain fused to the Cterminus of full length US9 in g9A10pep promotes APP α-cleavage, this way reducing the production of Aβ peptides and βAICD (β APP Intra-Cellular Domain). Due to the properties of US9, the functional outcome of the presence of g9A10pep can be as effective as the US9-retargeted α-secretase, and activity will be expressed in endosomes (the cellular compartment for APP β-cleavage) while endogenous ADAM10 is mostly present at the plasma membrane. Additionally, the phosphotyrosine binding domain (PTB) of X11 fused to N-terminal US9 trans-membrane domain (TM) in gPTB9TM alters APP processing in a different way. US9TM shows a more pronounced plasma membrane localization and in gPTB9TM this property is repurposed to withdraw bound APP from cellular locations in which β-secretase activity occurs. (FIG. 20C) g9A10pep and gPTB9TM are both able to dramatically reduce the APP β-cleavage. APP overexpression in HEK293T cells induces the appearance of the β-cleaved product. When APP is co-expressed together with g9A10pep or gPTB9TM, APP β-cleavage is reduced by more than 60% (bottom bands and quantitative analysis). (FIG. 20D) Similar results as those shown in (FIG. 20C) where obtained in the presence of overexpressed BACE1. HEK293T cells transfected with APP and BACE1 generate a large amount of βAICD. The co-expression of g9A10pep or gPTB9TM is able to dramatically reduce the β-secretase activity. In FIG. 20C and FIG. 20D, gfp is used as control for the specific effects of g9A10pep and gPTB9TM. Collectively, data shown here support the ability of US9 to alter APP amyloidogenic processing exploiting multiple mechanisms. Additionally, both N- and C-US9 termini can be used to construct chimeric effectors, providing proper membrane orientation and luminal/extracellular or cytoplasmic exposure of active domains.

FIGS. 21A-21B illustrate that US9-based molecular tools efficiently transduce cultured cells and primary neurons. gP2A9A10pep is a genetic construct in which gfp (g) is not fused to the chimeric US9-based effector protein (9A10pep). Instead, the two proteins are simultaneously and equivalently expressed, thanks to the presence of a 2A self-cleaving peptide. In FIG. 21A, HEK293T cells were transfected with gP2A9A10pep, and transfection efficiency was assessed by fluorescence microscopy 24 hours post transfection. (FIG. 21B) Rat cortical neurons were infected on DIV7 (Days In Vitro) with a lentiviral vector carrying gP2A9A10pep. Cells were fixed 7 days post infection, on DIV14, and imaged under a fluorescence microscope. All neurons were positively transduced, as assessed by the expression of gfp. In several experiments performed with this and other viral vectors, infection and subsequent US9-driven effector peptides expression were well tolerated by the neurons.

### DETAILED DESCRIPTION OF THE INVENTION

Misprocessing of the Amyloid Precursor Protein (APP) to form amyloid β-protein is associated with neuropathological conditions such as Alzheimer's Disease and HIV-Associated Neurocognitive Disorders (HAND). In the amyloidogenic pathway, the APP fragment generated by the interaction of APP with β APP cleaving enzyme 1 (BACE1) further undergoes additional processing by the γ-secretase presenilin-1 to yield amyloid β-peptides. Therefore, reduced accumulation of β-cleaved APP would reduce amyloid β-peptides.

The intracellular trafficking of the Herpes Simplex Virus US9 protein overlaps with that of the enzymatic machinery responsible of APP processing, and therefore it was thought that US9 could enable targeting the functional cargos to cellular regions in which APP and (BACE1) interact. Hence, in the present invention US9-based constructs were designed to alter the APP processing leading to amyloid β formation, in which either the full length US9 or its trans-membrane domain is covalently linked to an “effector” protein. These constructs localize to membrane rafts (where catabolic ceramide generation is believed to promote APP processing to amyloid β-protein) and alter APP processing in the cell.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “effector protein” refers to a protein capable of modulating enzyme activity, gene expression, and/or cell signaling.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “functionally active” as applied to a fragment of a protein or polypeptide refers to the fact that the fragment has substantially the same biological and/or functional activity as the protein or polypeptide itself. In certain embodiments, the functionally active fragment has at least and/or greater than about 50%, 60%, 70%, 80%, 90%, 95%, 96%, 97%, 98%, 99%, or 100% of the functional activity of the protein or polypeptide.

As used herein, the term “g9A10pep” refers to a construct composed of a reporter gfp (green fluorescent protein), full length Herpes Simplex Virus (HSV) US9, and an effector protein composed of the peptidase domain of A disintegrin and metalloproteinase 10 (ADAM 10).

As used herein, the term “gPTB9TM” refers to a construct composed of a reporter gfp (green fluorescent protein), an effector protein composed of the phosphotyrosine binding domain (PTB) of X11, and the HSV US9 trans-membrane domain (TM).

The terms “patient,” “subject” or “individual” are used interchangeably herein, and refer to any animal, or cells thereof whether in vitro or in situ, amenable to the methods described herein. In a non-limiting embodiment, the patient, subject or individual is a human. In other embodiments, the patient is a non-human mammal including, for example, livestock and pets, such as ovine, bovine, porcine, canine, feline and murine mammals. In yet other embodiments, the patient is an avian animal or bird. Preferably, the patient, individual or subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

A “therapeutic” treatment is a treatment administered to a subject who exhibits signs of pathology, for the purpose of diminishing or eliminating those signs.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

The following abbreviations are used herein: ADAM10=A disintegrin and metalloproteinase, APP=Amyloid Precursor Protein, BACE1=β APP cleaving enzyme 1, βAICD=β APP Intra-Cellular Domain, HSV=Herpes Simplex Virus, PEG=polyethylene glycol, PNA=peptide nucleic acid, PTB=phosphotyrosine binding domain, TM=HSV US9's transmembrane domain.

### Construct

In one embodiment, the invention provides a construct comprising an at least one effector protein or a functionally active fragment thereof and a Herpes Simplex Virus (HSV) US9 protein or a functionally active fragment thereof. In certain embodiments, the effector protein or the functionally active fragment thereof is covalently coupled to the HSV US9 protein or the functionally active fragment thereof. When an effector protein is covalently coupled to a second protein, in various embodiments, there is a covalent bond between a N- or C-terminal residue in the effector protein and the C- or N-terminal residue in a second protein, respectively.

In certain embodiments, the effector protein comprises a non-amyloidogenic APP-cleaving protease. In certain embodiments, the non-amyloidogenic APP-cleaving protease is the peptidase domain of A disintegrin and metalloproteinase (ADAM10).

In certain embodiments, the effector protein comprises a phosphotyrosine binding domain (PTB) of X11.

In certain embodiments, the HSV US9 protein fragment comprises the HSV US9 transmembrane domain (TM).

In certain embodiments, a fragment of the effector protein comprises a subsequence of the effector protein. In certain embodiments, the fragment comprising the subsequence of the effector protein is functionally active. In various embodiments, the fragment comprising the subsequence of the effector protein has at least, greater than, or less than about 50%, 60%, 70%, 80%, 90%, 95%, 96%, 97%, 98%, 99%, or 100% of the functional activity of the effector protein.

In various embodiments, the HSV US9 or a functionally active fragment thereof targets the effector protein or a functionally active fragment thereof to a cellular membrane where pathologic APP processing occurs. In certain embodiments, the effector protein or a functionally active fragment thereof is coupled to the N-terminus of the HSV US9 protein. In certain embodiments, the effector protein or a functionally active fragment thereof is coupled to the C-terminus of the HSV US9 protein. In certain embodiments, the effector protein or a functionally active fragment thereof is coupled to the N-terminus of the TM. In certain embodiments, the effector protein or a functionally active fragment thereof is coupled to the C-terminus of the TM.

In certain embodiments, when the effector protein or a functionally active fragment thereof is attached to the C-terminus of the HSV US9 or a functionally active fragment thereof, the effector protein or functionally active fragment thereof is exposed on the luminal/extracellular side of a cell. In certain embodiments, when the effector protein or a functionally active fragment thereof is attached to the N-terminus of HSV US9 or a functionally active fragment thereof, the effector protein or a functionally active fragment thereof is exposed on the cytoplasmic side of the cell.

In certain embodiments, the effector protein or a functionally active fragment thereof is coupled through a linker to the HSV US9 protein or a functionally active fragment thereof. In certain embodiments, the linker comprises a polyethylene glycol (PEG) chain, or a peptide, or a peptide nucleic acid (PNA). In certain embodiments, the linker peptide comprises less than 50 amino acids.

In various embodiments, the construct reduces APP β-amyloidogenic processing. In certain embodiments, and without being bound by theory, the construct reduces APP β-amyloidogenic processing by promoting APP α-cleavage, thereby reducing the production of amyloid β-peptides and βAICD (β APP Intra-Cellular Domain). In certain embodiments, the construct reduces APP β-amyloidogenic processing by withdrawing APP from cellular locations in which β-secretase activity occurs, thereby increasing the plasma membrane localization of APP, where APP can undergo APP α-cleavage by interacting with endogenous ADAM10.

In another embodiment, the constructs of the invention are encoded in an expression vector. In certain embodiments, the expression vector is selected from the group consisting of cosmids, plasmids, and viruses.

### Methods

In further embodiments, the invention provides a method of reducing levels of amyloid-β peptide by administering a composition that includes the construct of the invention to a subject in need thereof. In another aspect the invention provides a method of treating a neuropathological condition in a subject, by administering a composition that includes the construct of the invention to the subject in need thereof.

In one embodiment, the construct includes g9A10pep in which the ADAM10 peptidase domain is fused to the C-terminus of full length HSV US9, and the reduction of APP β-amyloidogenic processing is achieved by targeting α-secretase activity (peptidase domain) in cellular regions where β-secretase BACE1 is predominantly active, (and where endogenous ADAM10 is absent, such as for example, in endosomes. The g9A10pep shows more APP-specific effect than ADAM10 over-expression and therefore a higher impact on reducing amyloid β-protein production.

In another embodiment, the construct includes gPTB9TM, in which the PTB of X11 is fused to N-terminal of HSV US9 TM. In certain embodiments, the effector protein has no enzymatic activity, and without being bound by theory, the construct reduces APP β-amyloidogenic processing by withdrawing APP from cellular locations in which β-secretase activity occurs, thereby increasing the plasma membrane localization of APP, where APP can undergo APP α-cleavage by endogenous ADAM10. Advantageously, since no additional enzymatic activity is introduced into cells upon administering the gPTB9TM construct to the subject, the cellular function of cells in the subject are not affected adversely.

In certain embodiments, the construct is administered as part of a pharmaceutical composition that includes at least one pharmaceutically acceptable carrier.

In certain embodiments, the neuropathological conditions include, but are not limited to, Alzheimer's-associated or HIV-associated neurocognitive disorders.

In certain embodiments, the subject is a mammal. In other embodiments, the subject is human.

### Kit

In yet further aspect, the invention provides a kit comprising the pharmaceutical composition of the invention and an instructional material for use thereof, wherein the instructional material comprises instructions for using the pharmaceutical composition to reduce amyloid-β peptide formation in vivo.

In certain embodiments, the pharmaceutical composition of the invention is as describe elsewhere, herein.

### Pharmaceutical Compositions and Formulations

The invention also encompasses the use of pharmaceutical compositions of the invention to practice the methods of the invention.

Such pharmaceutical compositions may be provided in a form suitable for administration to a subject, and may comprise one or more pharmaceutically acceptable carriers, one or more additional ingredients, or some combination of these. The compositions of the invention may comprise a physiologically acceptable salt, such as a compound contemplated within the invention in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, intracranial, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one construct of the invention and a pharmaceutically acceptable carrier.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. The therapeutic formulations may be administered to the patient either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or condition in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, intracranial, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or diglycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction and assaying conditions with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

Sequences

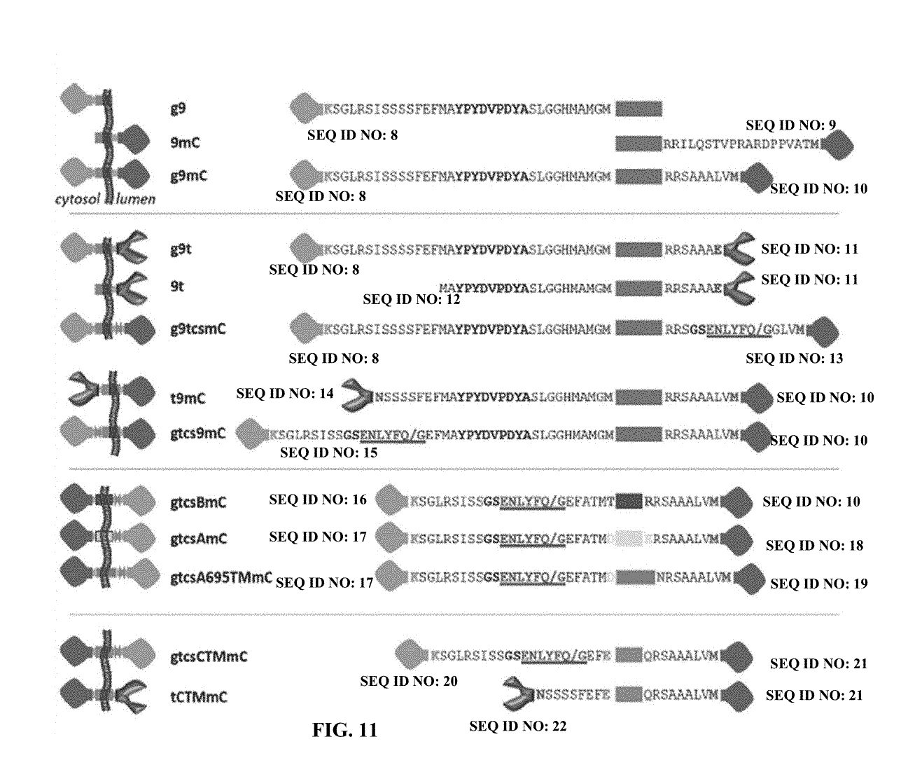

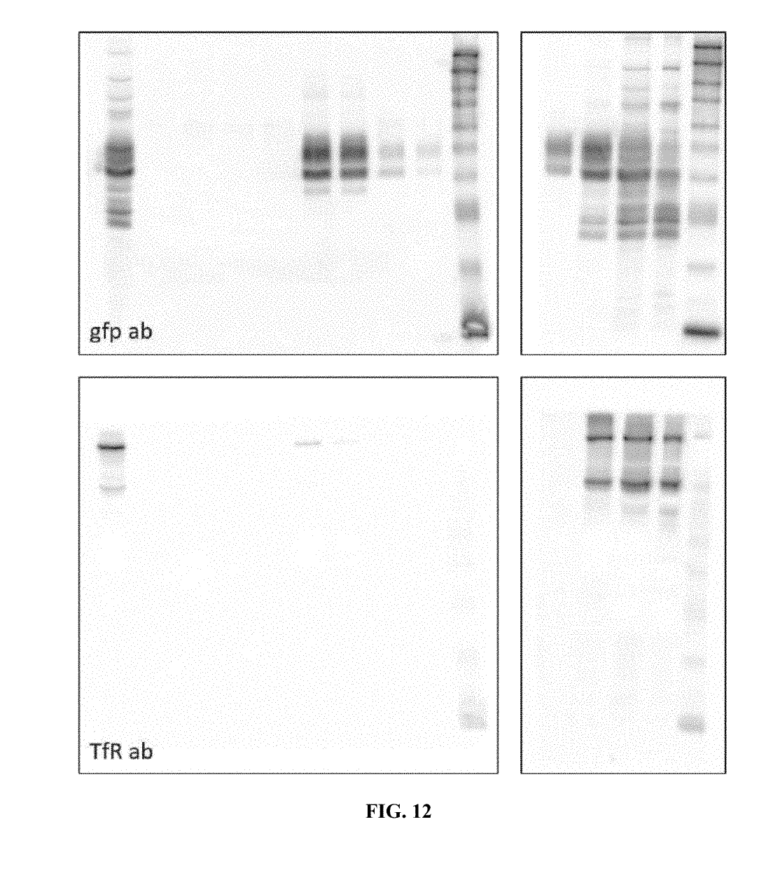

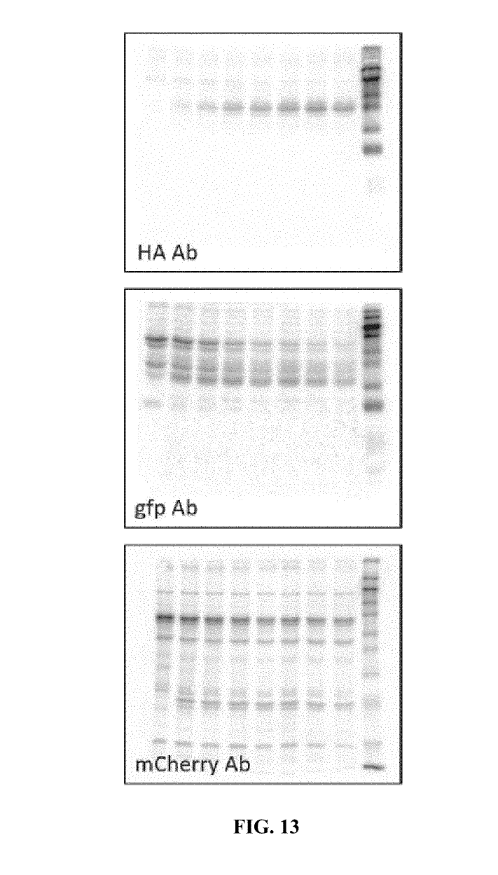

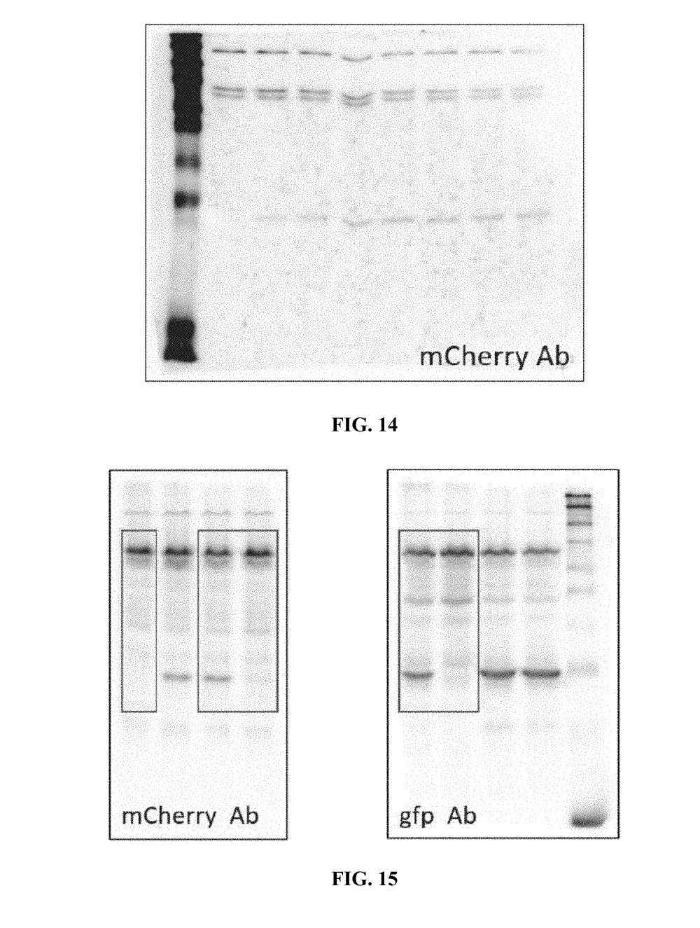

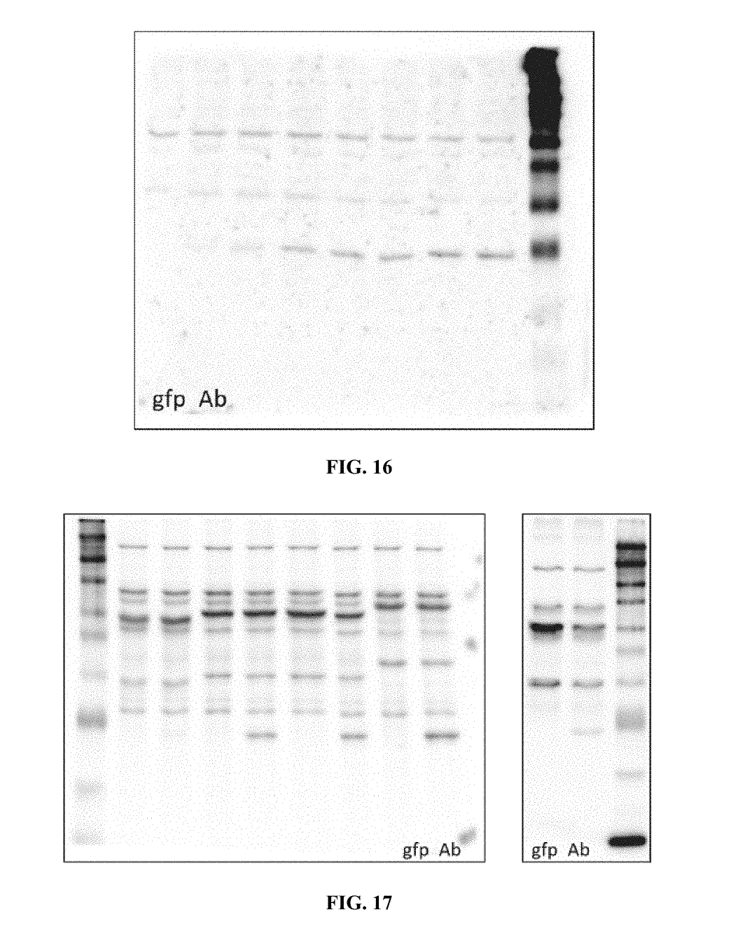

In various embodiments, a reporter gfp has the sequence of SEQ ID NO: 1:
MVSKGEELFTGVVPILVELD GDVNGHKFSV SGEGEGDATY GKLTLKFICT TGKLPVPWPT
LVTTLTYGVQ CFSRYPDHMK QHDFFKSAMP EGYVQERTIF FKDDGNYKTR AEVKFEGDTL VNRIELKGID
FKEDGNILGH KLEYNYNSHN VYIMADKQKN GIKVNFKIRH NIEDGSVQLA DHYQQNTPIG DGPVLLPDNH
YLSTQSALSK DPNEKRDHMV LLEFVTAAGI TLGMDELYKS GLRSRAQASN SAVDGTAGPG STGSR.
In various embodiments, an HSV US9 (HSV-1, strain 17) has the sequence of
SEQ ID NO: 2:
MTSRLSDPNSSARSDMSVPLYPTASPVSAEAYYSESEDEAANDFLVRMGRQQSVLRRRRRRTRCVGMVIACLLVAV
LSGGFGALLMWLLR.
In various embodiments, a peptidase (enzymatic) domain of ADAM10
(homo sapiens) has the sequence of SEQ ID NO: 3:
N TCQLYIQTDH LFFKYYGTRE AVIAQISSHV KAIDTIYQTT DFSGIRNISF MVKRIRINTT
ADEKDPTNPF RFPNIGVEKF LELNSEQNHD DYCLAYVFTD RDFDDGVLGL AWVGAPSGSS GGICEKSKLY
SDGKKKSLNT GIITVQNYGS HVPPKVSHIT FAHEVGHNFG SPHDSGTECT PGESKNLGQK ENGNYIMYAR
ATSGDKLNNN KFSLCSIRNI SQVLEKKRNN CFVESGQPI.
In various embodiments, ADAM10 (homo sapiens) has the sequence of
SEQ ID NO: 4:
MVLLRVLILL LSWAAGMGGQ YGNPLNKYIR HYEGLSYNVD SLHQKHQRAK RAVSHEDQFL RLDFHAHGRH
FNLRMKRDTS LFSDEFKVET SNKVLDYDTS HIYTGHIYGE EGSFSHGSVI DGRFEGFIQT RGGTFYVEPA
ERYIKDRTLP FHSVIYHEDD INYPHKYGPQ GGCADHSVFE RMRKYQMTGV EEVTQIPQEE HAANGPELLR
KKRTTSAEKN TCQLYIQTDH LFFKYYGTRE AVIAQISSHV KAIDTIYQTT DFSGIRNISF MVKRIRINTT
ADEKDPTNPF RFPNIGVEKF LELNSEQNHD DYCLAYVFTD RDFDDGVLGL AWVGAPSGSS GGICEKSKLY
SDGKKKSLNT GIITVQNYGS HVPPKVSHIT FAHEVGHNFG SPHDSGTECT PGESKNLGQK ENGNYIMYAR
ATSGDKLNNN KFSLCSIRNI SQVLEKKRNN CFVESGQPIC GNGMVEQGEE CDCGYSDQCK DECCFDANQP
EGRKCKLKPG KQCSPSQGPC CTAQCAFKSK SEKCRDDSDC AREGICNGFT ALCPASDPKP NFTDCNRHTQ
VCINGQCAGS ICEKYGLEEC TCASSDGKDD KELCHVCCMK KMDPSTCAST GSVQWSRHFS GRTITLQPGS
PCNDFRGYCD VFMRCRLVDA DGPLARLKKA IFSPELYENI AEWIVAHWWA VLLMGIALIM LMAGFIKICS
VHTPSSNPKL PPPKPLPGTL KRRRPPQPIQ QPQRQRPRES YQMGHMRR.
In various embodiments, a phosphotyrosine binding domain of X11 has the
sequence of SEQ ID NO: 5:
PEDLIDGII FAANYLGSTQ LLSDKTPSKN VRMMQAQEAV SRIKMAQKLA KSRKKAPEGE SQPMTEVDLF
ISTQRIKVLN ADTQETMMDH PLRTISYIAD IGNIVVLMAR RRMPRSNSQE NVEASHPSQD GKRQYKMICH
VFESEDAQLI AQSIGQAFSV AYQEFLRANG INP.
In various embodiments, X11 has the sequence of SEQ ID NO: 6:
MNHLEGSAEV EVTDEAAGGE VNESVEADLE HPEVEEEQQQ PPQQQHYVGR HQRGRALEDL RAQLGQEEEE
RGECLARSAS TESGFHNHTD TAEGDVIAAA RDGYDAERAQ DPEDESAYAV QYRPEAEEYT EQAEAEHAEA
THRRALPNHL HFHSLEHEEA MNAAYSGYVY THRLFHRGED EPYSEPYADY GGLQEHVYEE IGDAPELDAR
DGLRLYEQER DEAAAYRQEA LGARLHHYDE RSDGESDSPE KEAEFAPYPR MDSYEQEEDI DQIVAEVKQS
MSSQSLDKAA EDMPEAEQDL ERPPTPAGGR PDSPGLQAPA GQQRAVGPAG GGEAGQRYSK EKRDAISLAI
KDIKEAIEEV KTRTIRSPYT PDEPKEPIWV MRQDISPTRD CDDQRPMDGD SPSPGSSSPL GAESSSTSLH
PSDPVEASTN KESRKSLASF PTYVEVPGPC DPEDLIDGII FAANYLGSTQ LLSDKTPSKN VRMMQAQEAV
SRIKMAQKLA KSRKKAPEGE SQPMTEVDLF ISTQRIKVLN ADTQETMMDH PLRTISYIAD IGNIVVLMAR
RRMPRSNSQE NVEASHPSQD GKRQYKMICH VFESEDAQLI AQSIGQAFSV AYQEFLRANG INPEDLSQKE
YSDLLNTQDM YNDDLIHFSK SENCKDVFIE KQKGEILGVV IVESGWGSIL PTVIIANMMH GGPAEKSGKL
NIGDQIMSIN GTSLVGLPLS TCQSIIKGLK NQSRVKLNIV RCPPVTTVLI RRPDLRYQLG FSVQNGIICS
LMRGGIAERG GVRVGHRIIE INGQSVVATP HEKIVHILSN AVGEIHMKTM PAAMYRLLTA QEQPVYI.
In various embodiments, a trans-membrane domain of HSV US9 (HSV-1, strain 17)
has the sequence of SEQ ID NO: 7:
VIACLLVAVL SGGFGALLMWL.
SEQ ID NO: 8-SEQ ID NO: 22 are sequences for regions joining different
elements (gfp, mCherry, TEV protease, targeting domain) of the construct.
These sequences correspond to the sequences shown in FIG. 11.
SEQ ID NO: 8
KSGLRSISSS SFEFMAYPYD VPDYASLGGH MAMGM
SEQ ID NO: 9
RRILQSTVPR ARDPPVATM
SEQ ID NO: 10
RRSAAALVM
SEQ ID NO: 11
RRSAAAE
SEQ ID NO: 12
MAYPYDVPDY ASLGGHMAMG M
SEQ ID NO: 13
RRSGSENLYF QGGLVM
SEQ ID NO: 14
NSSSSFEFMA YPYDVPDYAS LGGHMAMGM
SEQ ID NO: 15
KSGLRSISSG SENLYFQGEF MAYPYDVPDY ASLGGHMAMG M
SEQ ID NO: 16
KSGLRSISSG SENLYFQGEF ATMT
SEQ ID NO: 17
KSGLRSISSG SENLYFQGEF ATMD
SEQ ID NO: 18
ERSAAALVM
SEQ ID NO: 19
NRSAAALVM
SEQ ID NO: 20
KSGLRSISSG SENLYFQGEF E
SEQ ID NO: 21
QRSAAALVM
SEQ ID NO: 22
NSSSSFEFE

In various embodiments, sequences having at least about 80%, 85%, 90%, 91%, 92%, 93%, 94%, 95%, 96%, 97%, 98%, or 99% sequence homology to any of the sequences of SEQ ID NO:1 to SEQ ID NO:7 can also be used to form the constructs described herein.

In various embodiments, two or more proteins with sequences of SEQ ID NO:1 to SEQ ID NO:7 can be connected end-to-end by fusion of the terminal residues of the N- and C-termini of the proteins to provide the constructs described herein. In various embodiments, fusion of the N- and C-termini includes a linker between the terminal residues of the N- and C-termini of the proteins. Suitable linkers include polyethylene glycol (PEG), a peptide, or a peptide nucleic acid (PNA). The length of the linker is chosen so that the functional activity of the fused proteins is not adversely affected. The PEG chain can be of formula —O—(CH₂CH₂—O)ₙ, where n is an integer from 1 to 100. In various embodiments, n is 1, 2, 3, 4, 5, 6, 7, 8, 9 or 10. Peptide linkers include poly-glycine linkers containing up to 50 glycine residues, poly-proline linkers containing up to 50 proline residues, or combinations thereof. Peptide linkers are not limited to glycine and proline residues, and any amino acid or sequence of amino acids can be used provided the functional activity of the fused proteins is not adversely affected.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Cells Cultures and Transfections.

Rat cortical neurons (RNs) were obtained from the brains of 17-18 day old rat embryos and cultured in Neurobasal medium containing B27 supplement (Gibco) as detailed by Sengupta et al. (Sengupta, R. et al., 2009 J. Neurosci. Off. J. Soc. Neurosci. 29, 2534-2544). and originally described by Brewer et al (Brewer, G. J et al, 1993, J. Neurosci. Res. 35, 567-576).

For transfections, RNs were seeded on poly-L-Lysine coated glass coverslips (35,000 cells/coverslip) in Neurobasal/B27 medium containing 2% horse serum for 2 hours. Coverslips were then washed and media replaced with Neurobasal/B27 media containing GlutaMAX, Glutamic Acid and Gentamycin. After 4 days, fresh medium not containing L-Glutamic acid was used to replace old medium. At 5 days in vitro (DIV5), half volume was removed and stored at 37° C., and replaced with fresh medium. After 6 hours, the medium was reduced to 0.6 mL and the removed volume pooled with the previously stored medium to make conditioned medium. Lipofectamine 2000 (Invitrogen)—DNA complexes (2 μL of Lipofectamine 2000 per transfection) were prepared in OptiMEM (Gibco) according to manufacturer directions, incubated for 5 min at room temperature in the dark, and transferred dropwise on coverslips. After 1 hour incubation at 37° C., transfection medium was removed and replaced with conditioned medium.

Human Embryonic Kidney Cells (HEK) 293T were grown in Dulbecco Modified Eagle Medium (DMEM) supplemented with 10% Fetal Calf Serum (FCS), and Gentamycin. For transfections, cells were seeded on 12 well plates, 160,000 cells/well, the day before treatment. Lipofectamine 2000—DNA complexes were prepared and added to cells as described above for RNs, and incubated for 2.5 hours before replacement with fresh medium. For co-transfections with variable amounts of plasmids, the total amount of DNA in each sample was kept constant by adding the empty pcDNA3.1 vector.

### Animals

Animals were used as a source of brain tissue to prepare neuronal cultures, following the recommendations in the Guide for the Care and Use of laboratory Animals of the National Institute of Health. This protocol for harvesting brain tissue was approved by the Institutional Animal Care and Use Committee of Drexel University (PHS Animals Welfare Assurance #A-3222-01), approved on Sep. 18, 2015 (permit #20439).

Fluorescence Microscopy Analysis and Immunodetection.

Cells were fixed in 4% paraformaldehyde 24 hours post transfection. For permeabilization, fixed cells were incubated with Phosphate Buffered Saline (PBS) containing 0.2% Triton X100, 1% Bovine Serum Albumine (BSA), 0.5% FCS for 30 minutes at Room Temperature. Non permeabilized cells were incubated with the same buffer without Triton X100. Permeabilized and non permeabilized cells were first incubated with the indicated antibodies for 40 min at RT, and subsequently with the appropriate secondary antibodies for 20′ at RT. Nuclear counter-staining was done via incubation of fixed cells with Hoechst 33342 (Molecular Probes—Invitrogen). Primary antibodies used for immunodetection were the anti-mCherry Rabbit polyclonal Antibody from Abcam (ab 183528), 1:1000 dilution, and the anti-Hemagglutinin (HA) mouse monoclonal antibody from Santa Cruz Biotechnology (sc-7392), 1:500 dilution. Secondary antibodies (Life Technologies) were Alexa Fluor 488 goat anti-rabbit for detection of mCherry, and Alexa Fluor 568 goat anti-mouse for detection of HA.

Confocal images were taken under a Zeiss Axio Imager.Z1m microscope equipped with Plan-apochromat 63x/1.4 oil DIC objective. Acquisition software was the provided AxioPlan LSM 510. All images were analyzed and assembled using Fiji. Images are representative of the original data and comply with the journal image processing policy.

### Protein Electrophoretic Analysis and Western Blotting.

Cells were harvested and lysed in RIPA buffer (150 mM sodium chloride, 1% Triton X-100, 0.5% sodium deoxycholate, 0.1% SDS (sodium dodecyl sulfate), 50 mM Tris, pH 8.0) containing proteases/phosphatases inhibitors for 15′ on ice with occasional vortexing, and centrifuged for 10 min at 4° C. at 10,000 rpm. Protein extracts in the supernatants were separated on denaturing 10% polyacrylamide gels (SDS Page) and transferred to a PVDF membrane for immunoblotting. Primary antibodies used were: anti-mCherry Rabbit polyclonal from Abcam (ab 183528), 1:2000, anti-HA mouse monoclonal from Santa Cruz Biotechnology (sc-7392), 1:1000, anti-gfp Rabbit polyclonal from Santa Cruz Biotechnology. (sc-8334), 1:500, anti-human Transferrin Receptor (TfR) mouse monoclonal from Invitrogen (13-6800), 1:1000. Secondary antibodies and detection reagents were from Pierce (SuperSignal West Femto kit).

Lipid Raft Flotation Assay.

Identification of lipid rafts through flotation on Optiprep™ sucrose gradient is a standard procedure, widely described in the literature (Simons, K. et al., 1997, Nature 387, 569-572; Brown, D. A. et al., 1998 Annu. Rev. Cell Dev. Biol, 14, 111-136; Hooper, N. M. Mol., 1999, Membr. Biol. 16, 145-156; Lingwood, D. et al, 2007 Nat. Protoc. 2, 2159-2165). Here the protocol used to study the association of PRV US9 with lipid rafts from infected cells was followed (Lyman, M. G., et al, 2008, PLoS, Pathog. 4, e1000065).

Briefly, cells were harvested, washed once on ice in PBS, lysed in 1 ml ice cold lysis buffer (1% Triton X100 in TNE: 25 mM Tris pH 6.8, 150 mM NaCl, 5 mM EDTA) with proteases/phosphatases inhibitors, homog-enized by passing 15 times through an 18-gauge needle, rocked for 30 min at 4° C., homogenized again (5 times through an 18-gauge needle), and finally mixed with 2 ml of ice-cold 60% Optiprep™ density gradient medium (Sigma-Aldrich). The gradient was prepared by placing the cells homogenate at the bottom of a Beckman SW41 ultracentrifuge tube and subsequently overlaying it with 5 ml of ice-cold 30% Optiprep in TNE and 4 ml of ice-cold 5% Optiprep in TNE. Samples were centrifuged at 34,200 rpm (200,000×g) at 4° C. for 20 hours. 1 ml fractions were collected from top and analyzed by SDS Page.

Plasmid Construction.

A complete list of plasmids generated in this study is provided in FIG. 11. In all constructs, protein expression is driven by the CMV promoter present in the original pEGFP-C1 vector (Clontech) that was used to create gfp-HSV US9 (g9). Constructs were sequenced and functionally validated. Restriction enzymes and Q5 High Fidelity DNA polymerase used for cloning were from New England Biolabs. Ligations were performed using DNA Ligation kit ver. 2 from TaKaRa. Oligonucleotides were from IDT. In order to allow expression of C-terminal fusion proteins, the stop codon at the end of the HSV US9 sequence was removed by PCR in the intermediate plasmid g9nostop. mCherry sequence was amplified from pmCherry-N1 (Clontech) and inserted in g9nostop to generate g9mC. 9mC was constructed by replacing the gfp sequence in the intermediate plasmid 9g with the mCherry sequence from pmCherry-N1.

TEV protease sequence was amplified from plasmid mCherry LD_0_TEV (Daringer, N. M., 2014, ACS Synth. Biol. 3, 892-902). and inserted in g9nostop to generate g9t. 9t was subsequently obtained by collapsing the region comprising gfp in g9t. t9mC was obtained by replacing the gfp sequence in g9mC with the TEV protease amplified sequence. To construct the HSVUS9-based TEV protease substrates, oligonucleotides coding the amino acid sequence GSENLYFQ/G were annealed in vitro and inserted in g9mC (TEV protease cleavage site is underlined; cleavage occurs between Q/G). Convenient restriction sites were added at the oligonucleotides 5′- and 3′-ends to allow cloning between HSV US9 and mCherry in g9tcsmC, or gfp and HSV US9 in gtcs9mC. DNA sequences coding for BACE1 (aa 454-481) and APP (aa 694-729) regions comprising transmembrane domains were amplified from 293T cells genomic DNA and used to replace the HSV US9 sequence in gtcs9mC, to obtain gtscBmC and gtcsAmC, respectively. To generate gtcsA695TMmC, the sequence encompassing trans membrane and cytosolic domains of APP was amplified from plasmid pCAX APP 695 (Addgene #30137) and inserted into gtcs9mC to replace the HSV US9 sequence. APP amino acids numbers refer to APP770.

To generate gtcsCTMmC—the substrate based on HSV glycoprotein C (gC) trans membrane domain (CTM)—the sequence coding for aa 478-504 (sequence reference GenBank: GU734771.1) of gC was amplified from viral DNA and cloned into gtcsBmC to replace the BACE1 TM portion of the substrate. The CTM-driven TEV protease was obtained by replacing the gfp-tcs sequence in gtcsCTMmC with the TEV protease sequence, and the resulting plasmid was named tCTMmC.

A detailed description of regions present between main elements in all these constructs is given in FIG. 11.

### Example 1: HSV US9 Efficiently Drives Recombinant Proteins to Either Leaflet of Cellular Membranes

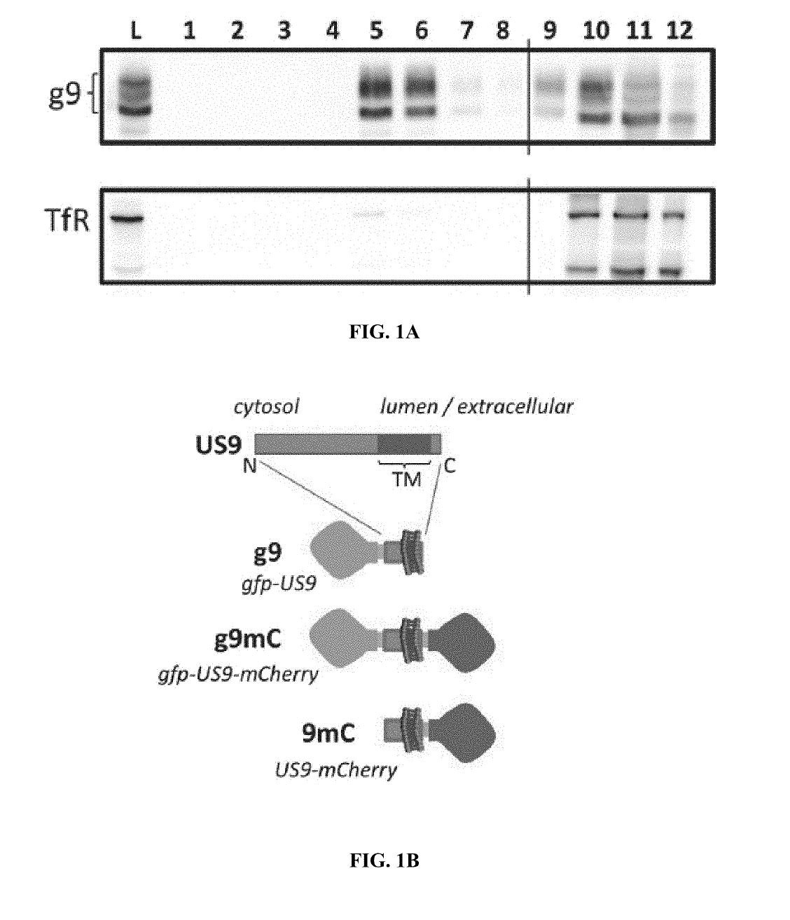

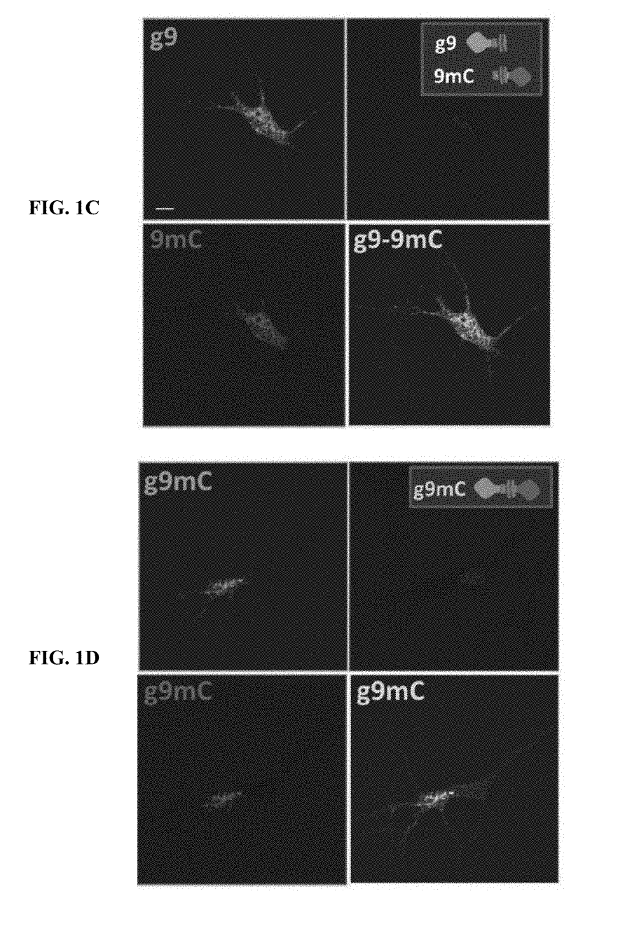

Modification of the HSV US9 N-terminus by the attachment of green fluorescent protein (gfp) does not alter its function in the context of viral infection. The gfp-HSV US9 fusion protein also retains its properties in the absence of other viral proteins and its natural cargo, the virion. Furthermore, HSV US9 from pseudorabies viruses (PRV) was found to associate with lipid rafts. To confirm that this property is maintained by gfp-HSV US9, its behavior on a sucrose gradient was analyzed (FIG. 1A), a well-established test assessing the ability of proteins to co-fractionate with lipid rafts (Simons, K. et al., 1997, Nature 387, 569-572; Brown, D. A. et al., 1998 Annu. Rev. Cell Dev. Biol, 14, 111-136; Hooper, N. M. Mol., 1999, Membr. Biol. 16, 145-156). Membrane proteins that are not solubilized, so called detergent resistant membranes, in the presence of cold non-ionic detergent such as Triton X-100 will float upward in a sucrose gradient and accumulate in fractions that are separated from those containing detergent sensitive membranes. This experiment showed that gfp-HSV US9 (i.e. g9) was highly enriched in fractions that corresponded to lipid rafts. In the same samples, the transferrin receptor, a transmembrane protein widely used as a non-lipid raft marker, was mostly found in detergent sensitive non-lipid raft fractions.

Constructs were generated using the fluorescent reporter protein mCherry attached to the C-terminus of HSV US9 (named 9mC). The mCherry sequence was also fused to gfp-HSV US9, to yield a double reporter of HSV US9 distribution that harbors two different fluorescent probes—i.e. on the N and C terminals of HSV US9 (g9mC). The topological organization of these constructs with respect to membrane insertion is shown in FIG. 1B. The HSV US9-mCherry construct (9mC) was expressed in primary neurons together with gfp-US9 (g9), and the results of this experiment are reported in FIG. 1C. The punctate staining of HSV US9-mCherry indicates a vesicular localization of the protein, scattered throughout the cytosol, with an increased accumulation in a region resembling the trans-Golgi network. This general distribution implied that mCherry fusion to HSV US9 C-terminus did not alter HSV US9-membrane insertion neither caused aggregation. More importantly, HSV US9-mCherry signal overlapped closely with that of gfp-HSV US9, indicating that N- or C-terminal modifications of HSV US9 are both compatible with HSV US9 targeting capabilities.

The ability of HSV US9 to act as a transport protein independently of the side on which the exogenous sequence is attached is further reinforced by the results obtained with the gfp-HSV US9-mCherry chimera (FIG. 1D). Although a HSV US9 marker to which it would have been possible to directly compare the fluorescent profile of g9mC was not available, the analysis of its distribution strongly resembles that of gfp-HSV US9. Additionally, no fusion protein aggregation or cell damage was detected, suggesting that the addition of the two fluorescent cargos does not interfere with the physiological properties of HSV US9.

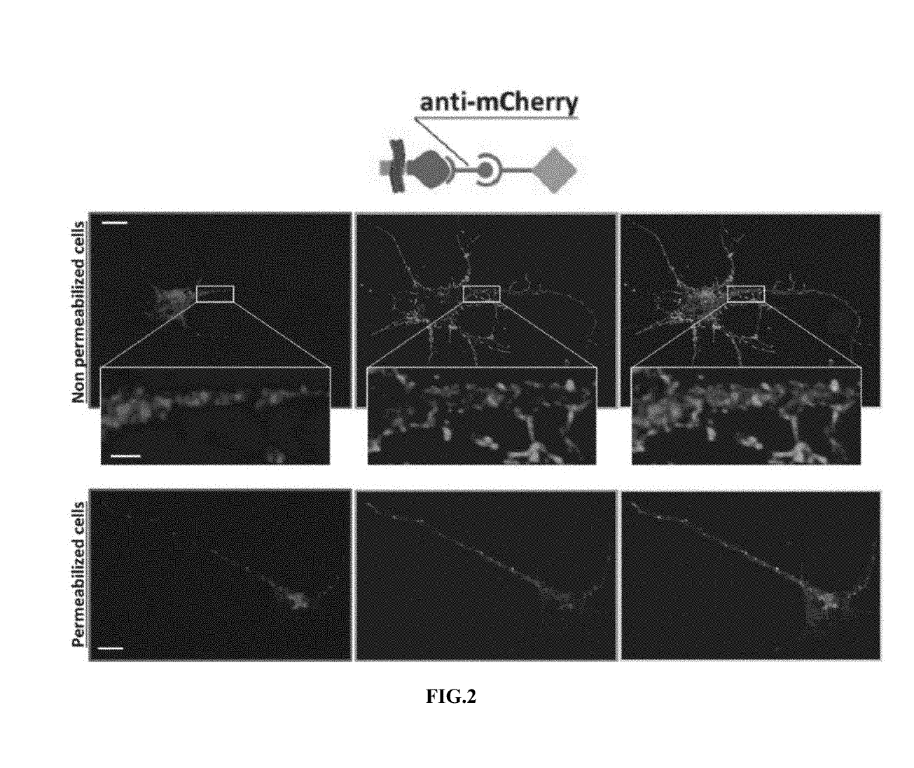

The correct orientation of the newly generated HSV US9-mCherry was additionally tested using immunofluorescence with an antibody directed against mCherry. In this orientation, mCherry is present on the non-cytosolic side of membranes, i.e. vesicle lumen and extracellular space. Therefore, if cells are not permeabilized, the plasma membrane should be labeled with fluorescent antibody while mCherry on vesicles should not be accessible. The results (FIG. 2, top panels) confirmed the correct orientation of HSV US9-mCherry expressed in rat neurons, with a typical plasma membrane staining with the mCherry antibody, and complete absence of immunofluorescent signal from mCherry-populated intracellular vesicles. The same vesicles became accessible to the antibody when cells were permeabilized (FIG. 2, bottom panels), with overlapping distributions of mCherry) and anti-mCherry (green) immunodetection. The cytosolic exposure of gfp in gfp-HSV US9 was confirmed by detection of the chimeric protein using an antibody against the hemagglutinin (HA) tag inserted between gfp and HSV US9. In the absence of membrane permeabilization, HA was not available and no signal was detected (FIG. 8). These results indicate that attachment of an exogenous sequence to either side of US9 does not alter its distribution in transfected cells. Consequently, HSV US9 is able to target exogenous proteins to both leaflets of cellular membranes.

### Example 2: Design, Construction and Analysis of a US9-Driven Functional Assay

The HSV US9 autonomous targeting properties described so far using confocal microscopy and biochemical means suggest that HSV US9 is well-suited for studies intended to analyze the intracellular trafficking and distribution of lipid-raft targeted proteins, such as those involved in APP processing. However, in order to demonstrate that HSV US9 can localize in close proximity of these proteins (i.e. sufficiently to interact with them as would be necessary for future interventional experiments using HSV US9-driven enzymes), a HSV US9-based functional assay was developed. In so doing, the HSV US9's ability to target a cargo that retains functional activity to specific cellular districts was also establish.

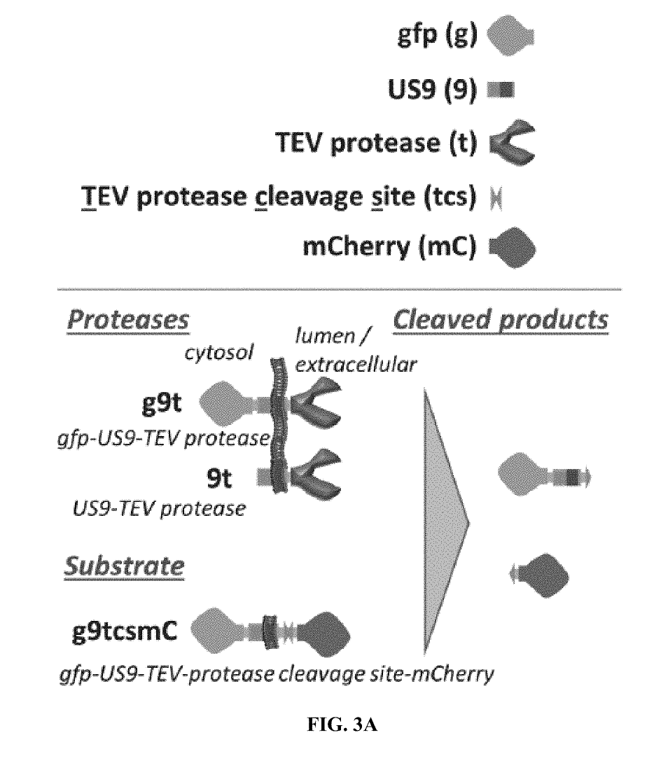

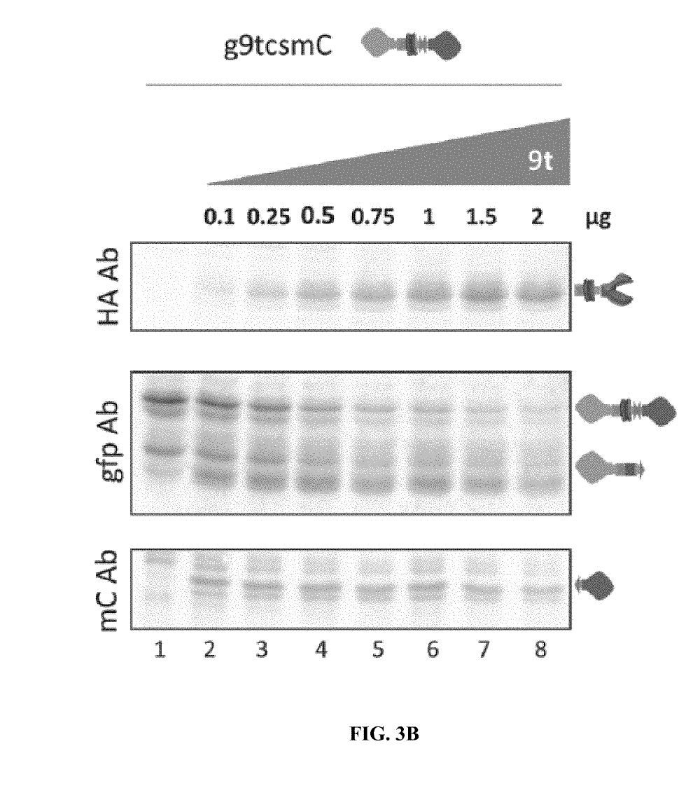

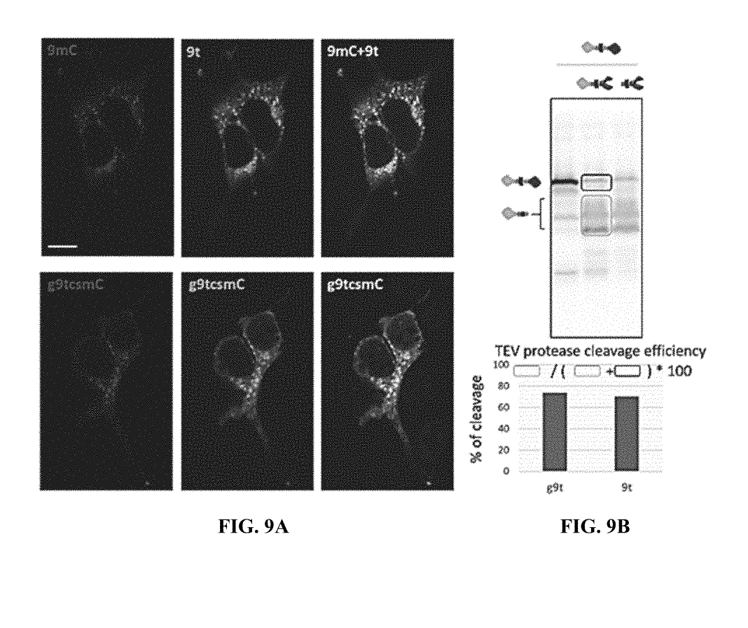

For the purpose of developing a functional assay, the 27 KDa catalytic domain of the Nuclear Inclusion a (NIa) protein from the Tobacco Etch Virus (TEV) was utilized. This enzymatic activity has been well-characterized in TEV and is not present in mammalian cells, providing a very clean system to detect HSV US9-driven activities. The short cleavage site recognized by the TEV protease is not targeted by other endogenous cellular enzymes, linking the generation of a cleaved product to the presence and close proximity of exogenous protease and substrate. The HSV US9-driven functional assay was assembled as described in FIG. 3A. It is composed of two different parts: a HSV US9-driven TEV protease, and a HSV US9-driven substrate. The HSV US9-driven TEV protease (named 9t) was constructed by attaching the TEV protease to the HSV US9 C-terminus. As a backbone for the substrate gfp-HSV US9-mCherry, modified by inserting the TEV protease cleavage sequence (tcs) between HSV US9 and mCherry (g9tcsmC) was used. The outcome of effective functional targeting produced two cleaved products from the TEV substrate parent protein with different sizes and recognizable and distinguishable with proper antibodies. The system was expressed and analyzed in HEK-293T cells and representative images of the distribution of HSV US9 driven protease and substrate are shown in FIG. 9A. The functional targeting of HSV US9-driven enzyme and substrate was tested by analyzing the presence of cleaved products in protein extracts from cells co-transfected with both constructs. As shown in FIG. 3B, the presence of HSV US9-driven protease (9t) in cells expressing HSV US9-driven substrate (g9tcsmC; lanes 2-8) correlates with the appearance of low molecular weight bands detected with proper antibodies (boxes gfp Ab and mCherry Ab).

The same bands were absent from cell lysates in which only the substrate was present (lane 1). The larger cleaved products detected with the gfp antibody is composed of the N-terminal portion of the substrate, containing gfp fused to HSV US9 (HSV US9 yields multiple bands in polyacrylamide gels, probably due to post-translational modifications). The smaller fragment corresponds to the residual C-terminal mCherry. The appearance of the cleaved fragments correlates with the reduction of the full-length uncleaved substrate and with increased expression of TEV protease (FIG. 3B, box HA Ab). The efficiency of the HSV US9-driven functional assay was around 70%. To account for possible variability in proteins expression due to different transfection efficiency across samples, the total amount of available substrate as the sum of uncleaved and cleaved fragments was defined, and these values measured in each individual sample were used to calculate the percentage of cleavage as follows; cleaved/(cleaved+uncleaved)*100 (FIG. 9B).

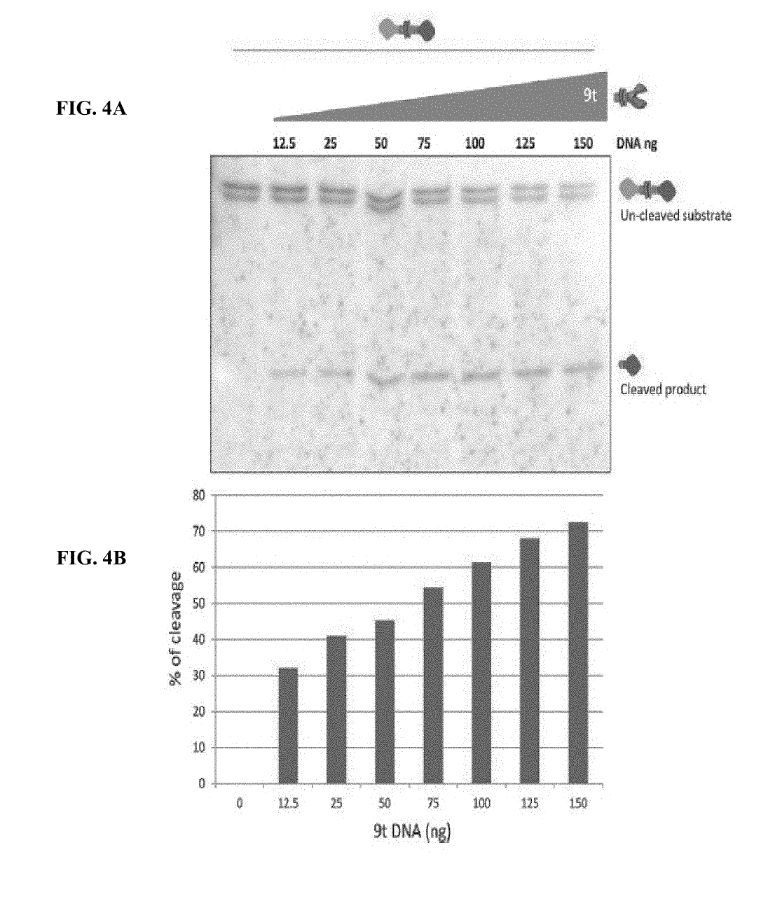

Cells were transfected with a constant amount of substrate and with increasing amounts of HSV US9-protease DNA (9t) ranging from 0 to 150 ng. No cleaved fragments were produced in the absence of the protease (first lane in FIG. 4A). The increasing expression of HSV US9-driven TEV protease caused a corresponding increase in the cleavage of the substrate, clearly detected in lanes 2 (12.5 ng 9t) through 8 (150 ng9t). The quantitative analysis of the HSV US9-driven activity (FIG. 4B), calculated as explained elsewhere herein, showed a direct correlation between the expression of the protease in this range and the amount of cleaved product. In summary, the HSV US9-driven cleavage assay demonstrates the ability of HSV US9 to target a functional activity to specific cellular microdomains in a stringent and measurable way.

### Example 3: HSV US9-Driven Enzymes are Active in the Vesicle Lumen and Observe Membrane Leaflet Specificity

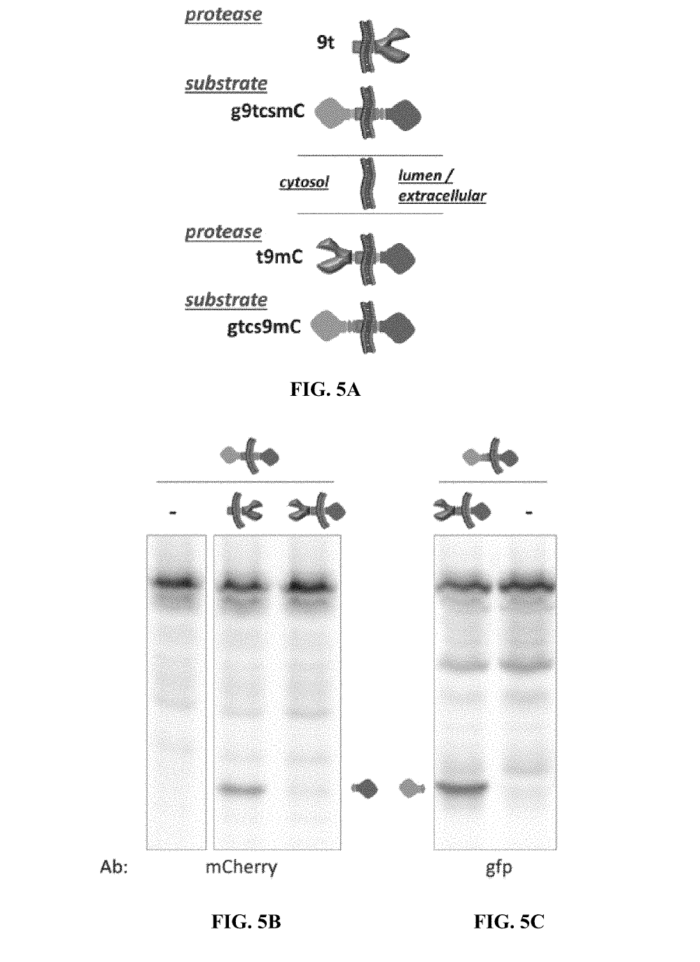

Similar to the evidence indicating that the membrane leaflet orientation of fused fluorescent proteins is determined by their addition to the C- or N-terminal of HSV US9 (FIGS. 1A-1D and 2), sequence-based structural prediction support the conclusion that molecules fused to the C-terminal domain of HSV US9 are exposed on the extracellular leaflet of the plasma membrane or in the lumen of intracellular vesicles. The effective cleavage of the HSV HSV US9-driven substrate by the protease depends on the close proximity and proper orientation of the two molecules. To further confirm that the co-presence of both substrate and protease in the same compartmentalized regions (lumen of transport vesicles or extra-cellular space) dictates functional activity. It was reasoned that the presence of the enzyme close to its substrate but exposed on the cytosolic side of vesicles would be ineffective toward the HSV US9-driven substrate (g9tcsmC) if the cleavage site is compartmentalized in the vesicle lumen. Therefore, a novel HSV US9-driven protease was generated by replacing gfp with the TEV protease in the gfp-HSV US9-mCherry construct (FIG. 5A) and named it t9mC. In this orientation, the protease hangs on the cytosolic leaflet of vesicles and plasma membranes. The same substrate previously used in the experiment shown in FIGS. 3A-3B and FIGS. 4A-4B (i.e. with the cleavage site inside the lumen of the vesicle) was transfected alone or together with HSV US9-TEV protease or TEV protease-HSV US9-mCherry, and the samples were analyzed for the presence of a cleaved product (FIG. 5B). The enzyme was active toward its substrate when they were brought in close proximity by the targeting properties of HSV US9 and both oriented in the same way, i.e. exposed in the vesicles lumen. The protease oriented in the opposite manner—though still properly targeted by HSV US9—was completely ineffective and no cleaved fragment could be detected in cells co-transfected with g9tcsmC and t9mC. As a control for the correctness of the constructs used here, the functionality of the HSV US9-driven, cytosol-oriented TEV protease toward a HSV US9-substrate was also tested with the cleavage site exposed on the same side of vesicles membranes (as visually represented in FIG. 5A). The results of this experiment, shown in FIG. 5C, confirmed the ability of the enzyme to cleave a substrate that is both close and exposed on the same leaflet of membranes. Therefore, both close proximity and correct membrane orientation are achieved by the US9-driven functional assay.

### Example 4: The HSV US9-Driven TEV Protease Activity Occurs in Lipid Rafts

The HSV US9-dependent transport of virions or viral components in the context of viral infection relies on its ability to associate with lipid rafts. For HSV US9 to be used as an effective targeting tool, the chimeric molecules generated should functionally target lipid raft associated molecules. As a way to test that HSV US9 functionally localizes to relevant lipid raft-associated proteins, the HSV US9-driven protease from the functional assay was used in combination with protease substrates driven by the transmembrane domains of proteins involved in APP processing (i.e. APP and BACE1). APP processing to amyloid β-protein through sequential cleavage of APP is modulated by dynamic association with lipid rafts, and is a pathway highly relevant to pathology in neurons.

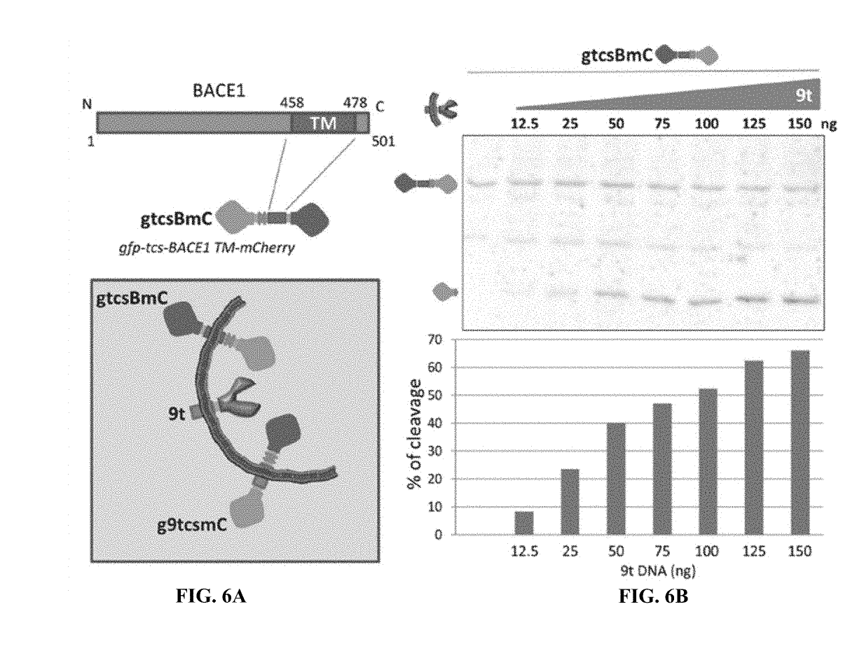

Briefly, APP can be processed by a β-secretase, BACE1, to produce a fragment that can then be cleaved by a γ-secretase to release amyloid β-protein. APP cleavage by BACE1 is thought to occur mainly in lipid rafts and BACE1 was selected as an indicator of the HSV US9-driven functional ability to affect lipid rafts molecular events. Hence, the HSV US9-based substrate g9tcsmC was modified by replacing the HSV US9 domain of the chimeric protein with the trans-membrane domain of BACE1 (BACE1-TM), to determine if it was accessible to the activity of the HSV US9-driven protease. Transmembrane domains play a critical role in proper membrane partitioning and proteins sorting/localization. Unlike HSV US9, BACE1 is a type I membrane protein, with the C-terminal portion exposed on the cytosolic side of membranes. Therefore, the orientation of the cleavable reporter was inverted, with the TEV protease cleavage site inserted between gfp and BACE1-TM, to generate gtcsBmC, as described in FIG. 6A.

Cells were co-transfected with the BACE1-based substrate and increasing amounts of the HSV US9-driven protease, and the activity of the enzyme was assessed. As shown in FIG. 6B, no cleavage occurred in the absence of the protease. When the enzyme was co-expressed with the substrate, a cleaved fragment was readily produced, in a HSV US9-protease expression dependent manner, as quantified in the histogram of FIG. 6B. The results of this experiment showed that a BACE1-based substrate is accessible to the activity of the HSV US9-driven protease.

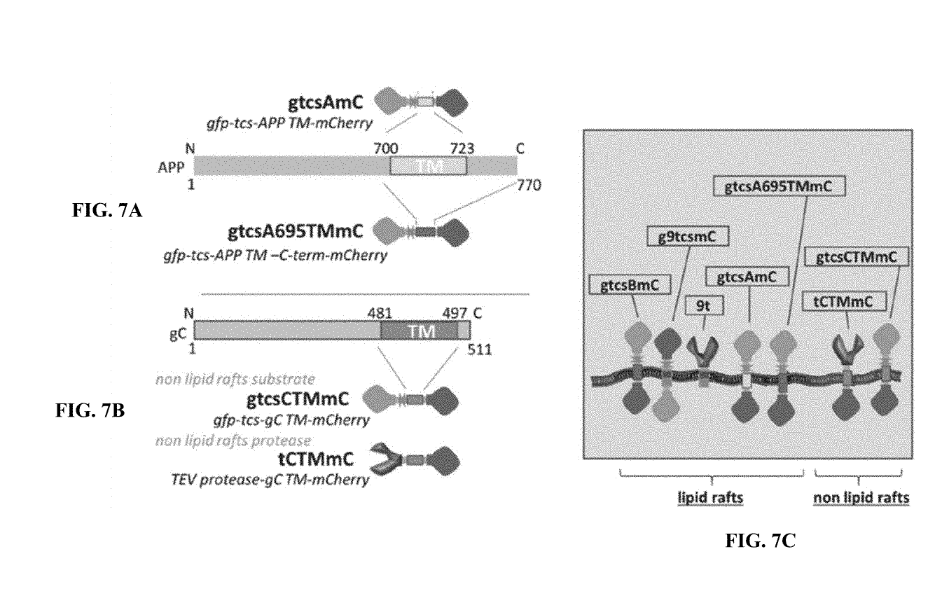

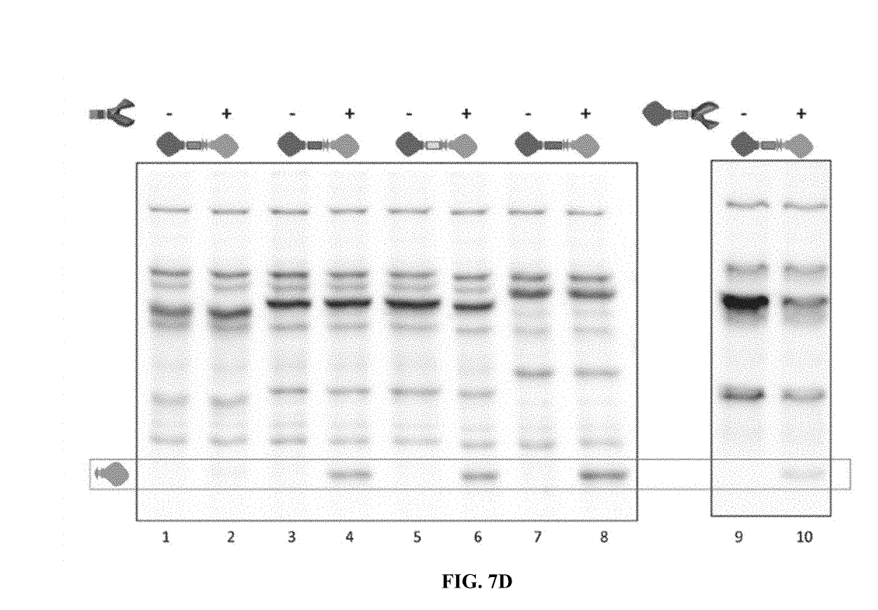

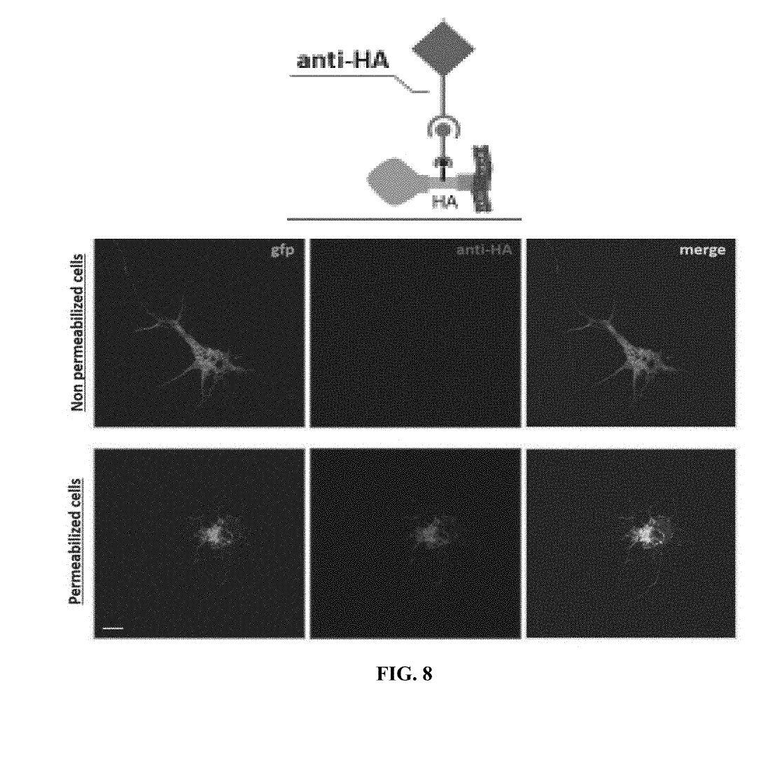

The other key component of the APP processing is APP itself. APP cleavage is a complex regulated process in which lipid rafts association seems to play a critical role. Attracted by the possibility that it might be possible to eventually modify the molecular machinery responsible for amyloid β-protein production, further investigated was the ability of HSV US9 to functionally act on APP-based substrates. APP is a type I membrane protein, and therefore gtcs-BmC was used as a backbone to replace the BACE1-TM with the APP trans-membrane domain, without or with the APP C-terminal domain. The two new substrates were named gtcsAmC and gtcsA695TMmC, respectively, and their organization with respect to the APP sequence is exemplified in FIG. 7A.

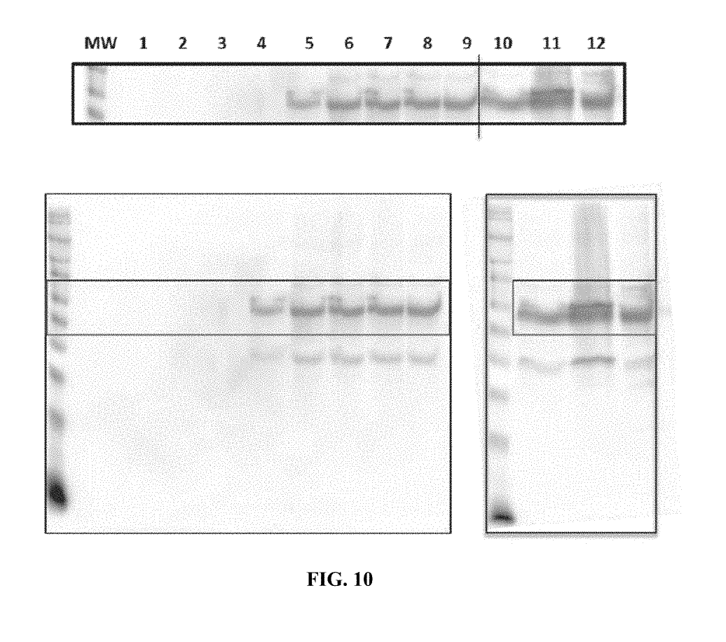

To add further evidence that HSV US9 fusion proteins localize specifically with other lipid raft proteins, additional functional experiments were performed using glycoprotein C (gC) from HSV 1. The gC is a type I membrane protein, similarly to BACE1 and APP, that does not localize to lipid rafts, and its trans-membrane domain (gC-TM) has been used in chimeric reporters to show non lipid raft localization. As shown in FIG. 7B, the gC-TM domain was inserted to replace the BACE1-TM in the BACE1-based substrate, and the resulting gC-based substrate gtcsCTMmC was used in HSV US9-driven functional assay (see FIG. 10 for the gtcsCTMmC's sucrose gradient validation). Finally, as a control for a non-lipid raft activity, the TEV protease was attached to the same gC-TM domain, and generated tCTMmC. The predicted localization of the polypeptides with respect to lipid rafts association is summarized in FIG. 7C.

These constructs were then tested for their ability to act as substrates for the activity of the HSV US9-driven protease (FIG. 7D). The gC-driven substrate was not cleaved when co-expressed together with 9t, implying a different membrane localization for the two proteins. On the other hand, substrates driven by BACE1-TM, APP-TM, or APP695TM could all interact with the protease, and the effect of this interaction resulted in the release of the cleaved fragment, detected with the gfp antibody in FIG. 7D (boxed bands). The non-lipid rafts gC-TM-driven substrate was cleaved in the presence of the gC-TM-driven protease, demonstrating that the absence of processing in cells co-expressing gtcsCTMmC and 9t was dependent on the different membrane localization of the two molecules.

### Example 5

Membrane microdomains (e.g. lipid rafts) and compartmentalization play a major role in numerous physiological and pathological processes, including HIV infection. Lipid rafts were recently suggested to also contribute to HIV neuropathogenesis. However, due to the limited availability of specific study tools, the exact contribution of lipid rafts to HAND is still unclear. The evidence that the lipid-raft dwelling protein HSV US9 can be used to investigate the contribution of lipid rafts-dependent changes induced by viral neurotoxins has been presented herein.

HSV US9 is an exogenous protein, with no known deleterious effect on mammalian cells. It is shown here that US9-driven recombinant cargos can be attached to either terminus of HSV US9 without altering its cellular distribution. Of note, HSV US9 distribution/trafficking behavior is maintained in different cell types, both primary cells (neurons) and various cell lines. The HSV US9-driven functional assay introduced here is based on the constitutively active exogenous proteolytic enzyme TEV protease. Interactions between the HSV US9-guided exogenous protease and recombinant proteins containing its cleavage sequence occurred predictably and relied on both protease and substrate sharing similar cellular compartmentalization, lateral membrane segregation, and membrane leaflet orientation. The observed cleavage of the substrates by the HSV US9-driven protease can only result from the close proximity of the two molecules. Therefore, cleavage represents the evidence of effective enzymatic processing, and provides corroborative functional evidence of lipidraft determination by biochemical means.

The HSV US9-based functional assay designed and tested was instrumental in demonstrating the ability of HSV US9 to target a functional cargo to the luminal leaflet of cellular vesicles, and to actively target molecules involved in APP processing. For this reason, an exogenous proteolytic activity not present in mammalian cells was selected, in order to provide unambiguous results exclusively dependent on the effect of the protease on its specific substrate. Nevertheless, based on data collected, the HSV US9 targeting capabilities can be used to directly manipulate the APP processing machinery, to help understand the contribution of individual steps in neuronal alterations depending on the presence of viral neurotoxins. Additionally, fluorescently tagged HSV US9 proteins such as g9, 9mC, and g9mC can provide a snapshot of targeted interaction/cleavage. Intra-neuronal accumulation of amyloid β-protein has been widely reported in the HIV-infected brain. However, HIV is known to affect amyloidogenesis in different ways, and deposition of amyloid β-protein plaques is also often found in aging HIV-infected individuals. As both HSV US9 and APP machinery can transiently accumulate on the plasma membrane, HSV US9-dependent functional activity can be used to study events that occur on the plasma membrane.

### Example 6: HSV US9-Based Strategies for Modulation of APP Processing

As stated elsewhere herein, the APP misprocessing to amyloid β formation is associated with neuropathological conditions, such as Alzheimer's Disease and HIV-Associated Neurocognitive Disorders (HAND). The intracellular trafficking of the Herpes Simplex Virus HSV US9 protein overlaps with that of the enzymatic machinery responsible of APP processing, and HSV US9 is able to target functional cargos to cellular districts in which APP and BACE1 ((3 APP cleaving enzyme 1) interact. Both HSV US9 and APP processing machinery rely on their ability to shuttle between endosomes and plasma membranes, as well as on their lateral accumulation in lipid rafts.

With the intent of altering APP processing that leads to amyloid β formation, a series of HSV US9-based chimeric proteins, in which either the full length HSV US9 or just its trans-membrane domain was fused to an “effector” protein, were generated.

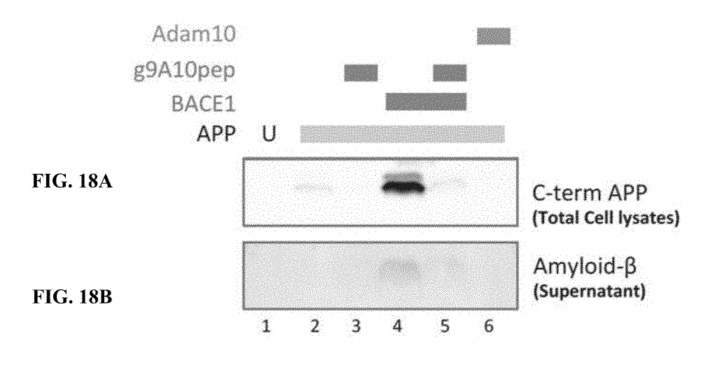

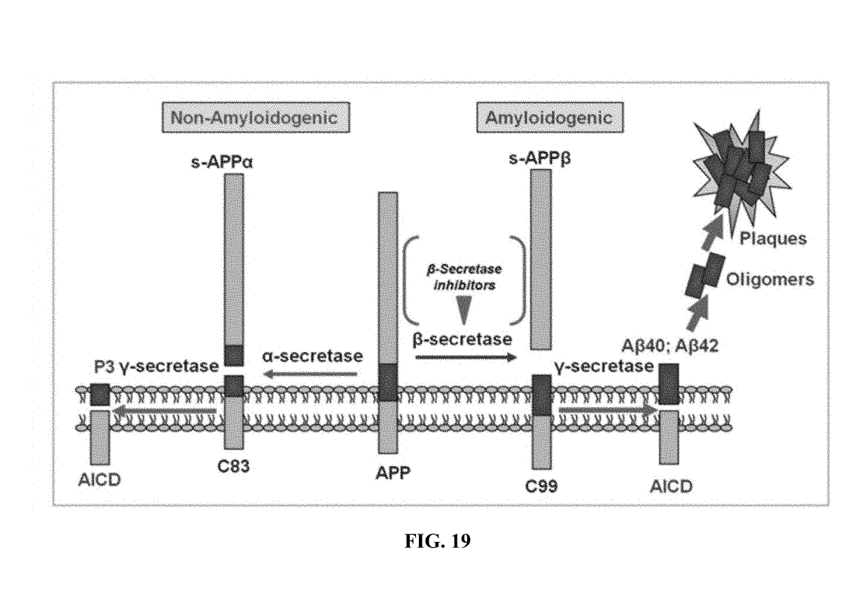

The chimeric protein g9A10pep—comprising a reporter gfp (green fluorescent protein), full length HSV US9, and the peptidase domain of the cellular α-secretase ADAM10 (A disintegrin and metalloproteinase 10)—was studied. Expression of this construct dramatically reduces the production of the APP C-terminal fragments, which result from APP β-cleavage by BACE1 (FIG. 18A). In the amyloidogenic pathway, additional processing of the APP fragment by the γ-secretase presenilin-1, follows BACE1 cleavage—yielding β-amyloid peptides. Thus, reduced accumulation of β-cleaved APP by g9A10pep activity can reduce amyloid-0 peptides.

Indeed, extracellular release of amyloid-0 from cells overexpressing APP and BACE1 in the presence of g9A10pep was almost undetectable (FIG. 18B). These data suggests that HSV US9-driven molecules can positively affect APP processing by counteracting the effect of cellular enzymes leading to the amyloidogenic pathway. Therefore, HSV US9 can be used to target and positively manipulate APP processing. In addition to g9A10pep, additional HSV US9-based constructs, in which different domains of ADAM10 were fused to HSV US9 C-terminus, were generated.

### Example 6: HSV US9-Based Molecular Tools can Reduce APP β-Amyloidogenic Processing by Exploiting Multiple Molecular Mechanisms

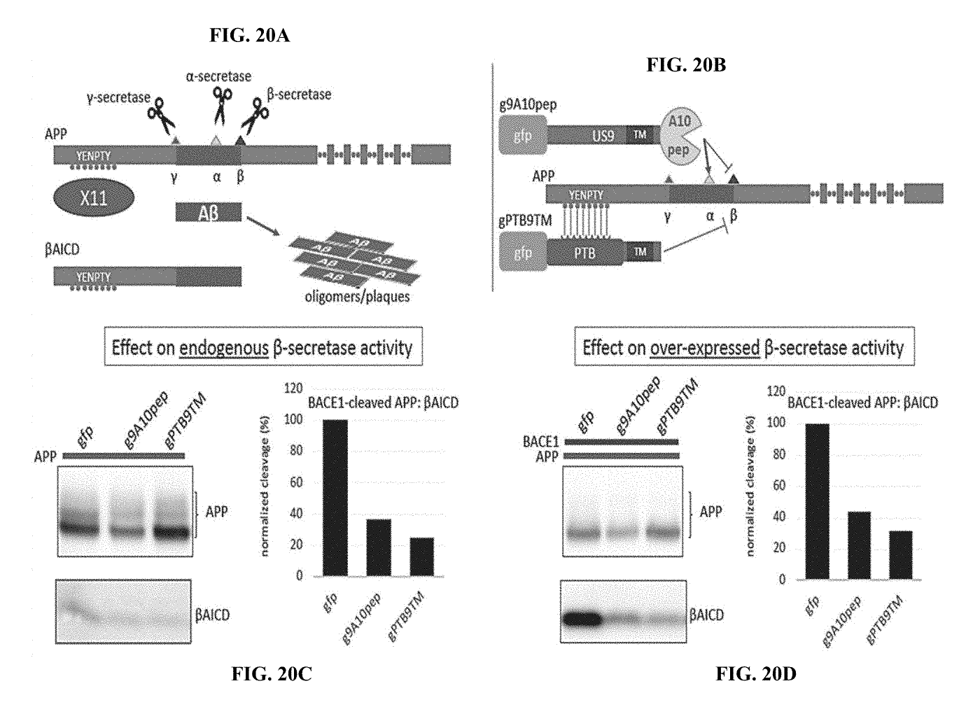

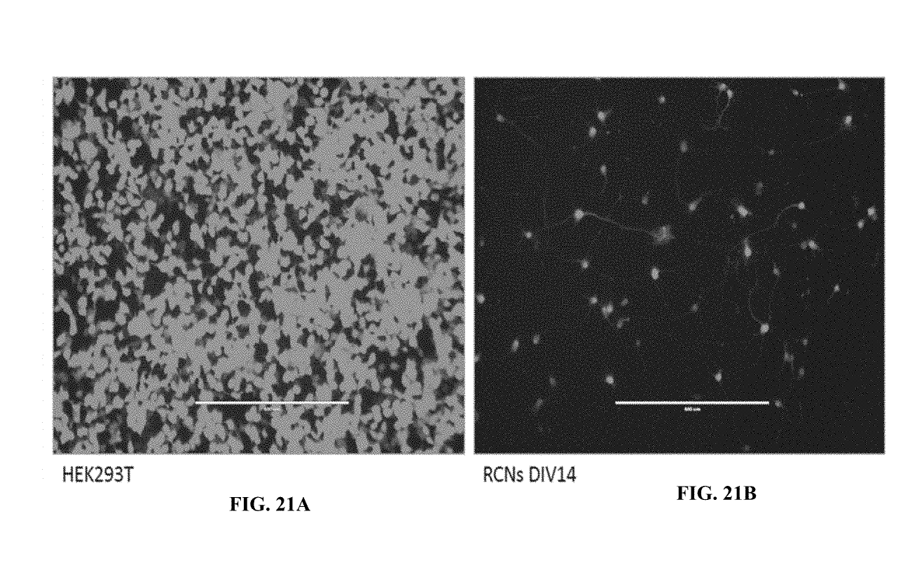

As shown in FIG. 20C, when APP is co-expressed together either with g9A10pep or gPTB9TM, APP β-cleavage is reduced by more than 60%. In g9A10pep construct (wherein, the ADAM10 peptidase domain fused to the C-terminus of full length HSV US9), the effector protein is the enzymatic domain of ADAM10, and the reduction of APP β-amyloidogenic processing is achieved by targeting the α-secretase activity (peptidase domain) in cellular districts where β-secretase BACE1 is predominantly active (recycling endosomes), and where endogenous ADAM10 is absent. The g9A10pep shows more APP-specific effect than ADAM10 over-expression and therefore a higher impact on reducing Aβ production.

In the gPTB9TM construct (wherein, the phosphotyrosine binding domain (PTB) of X11 fused to N-terminal HSV US9 trans-membrane domain (TM)), the effector protein has no enzymatic activity and the reduction of APP β-amyloidogenic processing is achieved by driving APP away from BACE1-enriched domains as result of PTB binding. The gPTB9TM increases plasma membrane localization of APP (and maybe favor APP interaction with ADAM10 on the cell surface, thereby reducing the levels of amyloid-β peptide by APP α-cleavage from endogenous ADAM10).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A construct comprising:
an effector protein or a functionally active fragment thereof;
a Herpes Simplex Virus (HSV) US9 protein or a functionally active fragment thereof, and
wherein the effector protein or the functionally active fragment thereof is covalently coupled to the HSV US9 protein or the functionally active fragment thereof.

2. The construct of claim 1, wherein the effector protein comprises a non-amyloidogenic Amyloid Precursor Protein (APP)-cleaving protease.

3. The construct of claim 2, wherein the effector protein comprises a peptidase domain of A disintegrin and metalloproteinase (ADAM10).

4. The construct of claim 1, wherein the effector protein comprises a phosphotyrosine binding domain (PTB) of X11.

5. The construct of claim 1, wherein the HSV US9 protein fragment comprises a HSV US9 transmembrane domain (TM).

6. The construct of claim 1, wherein the effector protein or functionally active fragment thereof is covalently coupled to the N-terminus of the HSV US9 protein.

7. The construct of claim 1, wherein the effector protein or functionally active fragment thereof is covalently coupled to the C-terminus of the HSV US9 protein.

8. The construct of claim 5, wherein the effector protein or functionally active fragment thereof is covalently coupled to the N-terminus of the TM.

9. The construct of claim 5, wherein the effector protein or functionally active fragment thereof is covalently coupled to the C-terminus of the TM.

10. The construct of claim 1, wherein the effector protein or functionally active fragment thereof is covalently coupled to the HSV US9 protein or functionally active fragment thereof through a linker.

11. The construct of claim 10, wherein the linker comprises a polyethylene glycol (PEG) chain, a peptide, or a peptide nucleic acid (PNA).

12. The construct of claim 11, wherein the peptide comprises less than 50 amino acids.

13. The construct of claim 1, wherein the construct reduces APP β-amyloidogenic processing.

14. The construct of claim 13, wherein the APP β-amyloidogenic processing is reduced by promoting APP α-cleavage.

15. The construct of claim 13, wherein the APP β-amyloidogenic processing is reduced in a cell by targeting APP away from β APP cleaving enzyme 1 (BACE1)-enriched domains and towards the plasma membrane of the cell to promote APP α-cleavage by endogenous ADAM10.

16. The construct of claim 1, wherein the effector protein or functionally active fragment thereof is exposed on the luminal/extracellular side of a cell.

17. The construct of claim 1, wherein the effector protein or functionally active fragment thereof is exposed on the cytoplasmic side of a cell.

18. An expression vector encoding the construct of claim 1.

19. The expression vector of claim 18, wherein the expression vector is selected from the group consisting of cosmids, plasmids, and viruses.

20. A method of reducing levels of amyloid β-peptide in a subject, the method comprising administering a therapeutically effective amount of a pharmaceutical composition comprising the construct of claim 1 to a subject in need thereof.

21. A method of treating a neuropathological condition in a subject, the method comprising administering a therapeutically effective amount of a pharmaceutical composition comprising the construct of claim 1 to a subject in need thereof.

22. The method of claim 23, wherein the neuropathological condition comprises an Alzheimer's-associated disorder, an HIV-associated neurocognitive disorders, or combinations thereof.

23. The method of claim 20, wherein the subject is a mammal.

24. The method of claim 24, wherein the subject is human.

25. A kit comprising the pharmaceutical composition of claim 22 and an instructional material for use thereof, wherein the instructional material comprises instructions for using the pharmaceutical composition to reduce amyloid-β peptide formation in vivo.


SEQUENCE LISTING (22 sequences)

SEQ ID NO: 1 - protein, 265 aa, Artificial Sequence
  synthetic sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSAVDGTAGPGSTGSR

SEQ ID NO: 2 - protein, 90 aa, Homo sapiens
MTSRLSDPNSSARSDMSVPLYPTASPVSAEAYYSESEDEAANDFLVRMGRQQSVLRRRRRRTRCVGMVIACLLVAVLSGGFGALLMWLLR

SEQ ID NO: 3 - protein, 240 aa, Homo sapiens
NTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPI

SEQ ID NO: 4 - protein, 748 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 5 - protein, 172 aa, Homo sapiens
PEDLIDGIIFAANYLGSTQLLSDKTPSKNVRMMQAQEAVSRIKMAQKLAKSRKKAPEGESQPMTEVDLFISTQRIKVLNADTQETMMDHPLRTISYIADIGNIVVLMARRRMPRSNSQENVEASHPSQDGKRQYKMICHVFESEDAQLIAQSIGQAFSVAYQEFLRANGINP

SEQ ID NO: 6 - protein, 837 aa, Homo sapiens
MNHLEGSAEVEVTDEAAGGEVNESVEADLEHPEVEEEQQQPPQQQHYVGRHQRGRALEDLRAQLGQEEEERGECLARSASTESGFHNHTDTAEGDVIAAARDGYDAERAQDPEDESAYAVQYRPEAEEYTEQAEAEHAEATHRRALPNHLHFHSLEHEEAMNAAYSGYVYTHRLFHRGEDEPYSEPYADYGGLQEHVYEEIGDAPELDARDGLRLYEQERDEAAAYRQEALGARLHHYDERSDGESDSPEKEAEFAPYPRMDSYEQEEDIDQIVAEVKQSMSSQSLDKAAEDMPEAEQDLERPPTPAGGRPDSPGLQAPAGQQRAVGPAGGGEAGQRYSKEKRDAISLAIKDIKEAIEEVKTRTIRSPYTPDEPKEPIWVMRQDISPTRDCDDQRPMDGDSPSPGSSSPLGAESSSTSLHPSDPVEASTNKESRKSLASFPTYVEVPGPCDPEDLIDGIIFAANYLGSTQLLSDKTPSKNVRMMQAQEAVSRIKMAQKLAKSRKKAPEGESQPMTEVDLFISTQRIKVLNADTQETMMDHPLRTISYIADIGNIVVLMARRRMPRSNSQENVEASHPSQDGKRQYKMICHVFESEDAQLIAQSIGQAFSVAYQEFLRANGINPEDLSQKEYSDLLNTQDMYNDDLIHFSKSENCKDVFIEKQKGEILGVVIVESGWGSILPTVIIANMMHGGPAEKSGKLNIGDQIMSINGTSLVGLPLSTCQSIIKGLKNQSRVKLNIVRCPPVTTVLIRRPDLRYQLGFSVQNGIICSLMRGGIAERGGVRVGHRIIEINGQSVVATPHEKIVHILSNAVGEIHMKTMPAAMYRLLTAQEQPVYI

SEQ ID NO: 7 - protein, 21 aa, Homo sapiens
VIACLLVAVLSGGFGALLMWL

SEQ ID NO: 8 - protein, 35 aa, Artificial Sequence
  synthetic sequence
KSGLRSISSSSFEFMAYPYDVPDYASLGGHMAMGM

SEQ ID NO: 9 - protein, 19 aa, Artificial Sequence
  synthetic sequence
RRILQSTVPRARDPPVATM

SEQ ID NO: 10 - protein, 9 aa, Artificial Sequence
  synthetic sequence
RRSAAALVM

SEQ ID NO: 11 - protein, 7 aa, Artificial Sequence
  synthetic sequence
RRSAAAE

SEQ ID NO: 12 - protein, 21 aa, Artificial Sequence
  synthetic sequence
MAYPYDVPDYASLGGHMAMGM

SEQ ID NO: 13 - protein, 16 aa, Artificial Sequence
  synthetic sequence
RRSGSENLYFQGGLVM

SEQ ID NO: 14 - protein, 29 aa, Artificial Sequence
  synthetic sequence
NSSSSFEFMAYPYDVPDYASLGGHMAMGM

SEQ ID NO: 15 - protein, 41 aa, Artificial Sequence
  synthetic sequence
KSGLRSISSGSENLYFQGEFMAYPYDVPDYASLGGHMAMGM

SEQ ID NO: 16 - protein, 24 aa, Artificial Sequence
  synthetic sequence
KSGLRSISSGSENLYFQGEFATMT

SEQ ID NO: 17 - protein, 24 aa, Artificial Sequence
  synthetic sequence
KSGLRSISSGSENLYFQGEFATMD

SEQ ID NO: 18 - protein, 9 aa, Artificial Sequence
  synthetic sequence
ERSAAALVM

SEQ ID NO: 19 - protein, 9 aa, Artificial Sequence
  synthetic sequence
NRSAAALVM

SEQ ID NO: 20 - protein, 21 aa, Artificial Sequence
  synthetic sequence
KSGLRSISSGSENLYFQGEFE

SEQ ID NO: 21 - protein, 9 aa, Artificial Sequence
  synthetic sequence
QRSAAALVM

SEQ ID NO: 22 - protein, 9 aa, Artificial Sequence
  synthetic sequence
NSSSSFEFE